SEQUENCE LISTING (9 sequences)

SEQ ID NO: 1 - protein, 6 aa, Homo sapiens
QRRSPP

SEQ ID NO: 2 - protein, 30 aa, Artificial Sequence
  Description of Artificial Sequence Synthetic polypeptide
YGRKKRRQRRRGGLSSPLALPSQRRSPPEE

SEQ ID NO: 3 - protein, 30 aa, Artificial Sequence
  Description of Artificial Sequence Synthetic polypeptide
YGRKKRRQRRRGGLSSPLALPSGGGGGGEE

SEQ ID NO: 4 - protein, 71 aa, Homo sapiens
ESARIEDMRKRHVEVSQAPLPPAPAYLSSPLALPSQRRSPPEEPPDFCCPKCQYQAPDMDTLQIHVMECIE

SEQ ID NO: 5 - protein, 71 aa, Mus sp.
ESARIEDMRKRHVETPQPPLLPAPAHHSFHLALSNQRRSPPEEPPDFCCPKCQYQAPDMDTLQIHVMECIE

SEQ ID NO: 6 - protein, 71 aa, Bos sp.
ESARIEDMRKRHVEVSQPPLAPAPGHHSFHLNPSSQRRSPPDEPPKFCCPKCQYQAPDIDTLQIHVMECIE

SEQ ID NO: 7 - protein, 71 aa, Rattus sp.
ESARIEDMRKRHVETSQPPLLPAPAHHSFHLALSNQRRSPPEEPPDFCCPKCQYQAPDMDTLQIHVMECIE

SEQ ID NO: 8 - protein, 60 aa, Drosophila sp.
TASPLSSSRSNLREEQRPILDPTGASSRTSDTTLRCPICSKSFNALSVLQSHVNDCLDLD

SEQ ID NO: 9 - protein, 6 aa, Artificial Sequence
  Description of Artificial Sequence Synthetic 6xHis tag
HHHHHH

(19) United States
(12) Patent Application Publication
(10) Pub. No.: US 2024/0166703 A1
(43) Pub. Date: May 23, 2024

(54) ANTI-INFLAMMATORY PEPTIDE AND METHOD OF USE THEREOF

(71) Applicants: The Regents of the University of California, Oakland, CA (US); SAN DIEGO STATE UNIVERSITY (SDSU) FOUNDATION, SAN DIEGO, CA (US); THE UNITED STATES OF AMERICA AS REPRESENTED BY THE DEPARTMENT OF VETERANS AFFAIRS

(72) Inventors: Gourisankar GHOSH, La Jolla, CA (US); Sushil MAHATA, San Diego, CA (US); Tom HUXFORD, San Diego, CA (US)

(73) Assignees: The Regents of the University of California, Oakland, CA (US); SAN DIEGO STATE UNIVERSITY (SDSU) FOUNDATION, SAN DIEGO, CA (US); THE UNITED STATES OF AMERICA AS REPRESENTED BY THE DEPARTMENT OF VETERANS AFFAIRS, (US)

(21) Appl. No.: 18/550,536

(22) PCT Filed: Mar. 30, 2022
(86) PCT No.: PCT/US2022/022425

§ 371 Date: Sep. 14, 2023

Related U.S. Application Data
(60) Provisional application No. 63/170,068, filed on Apr. 2, 2021.
(60) Provisional application No. 63/230,935, filed on Aug. 9, 2021.

(51) Int. Cl. C07K 14/47

(52) U.S. Cl. CPC C07K 14/4705

(57) ABSTRACT

The present disclosure provides a novel IKK2 inhibitor that does not target the kinase domain of IKK2 molecule. The novel IKK2 inhibitor is a cell penetrating NEMO^{ActPep} comprising a small segment (residues 384-389) of human NEMO to mediate interaction with and promote activation loop phosphorylation of IKK2. The NEMO^{ActPep} is derived from the second interaction interface of NEMO:IKK2 that is dependent upon NEMO binding to linear polyubiquitin. Methods of use the novel IKK2 inhibitor, and composition thereof, for preventing and/or treating inflammation in various diseases involving IKK-NF-κB signaling pathways are also provided.

### CROSS-REFERENCE

This application claims the benefit of U.S. Provisional Application No. 63/170,068, filed Apr. 2, 2021 and U.S. Provisional Application No. 63/230,935, filed Aug. 9, 2021, the entire contents of each application are incorporated herein by reference.

### SEQUENCE LISTING

The instant application contains a Sequence Listing which has been submitted electronically in ASCII format and is hereby incorporated by reference in its entirety. Said ASCII copy, created on Mar. 29, 2022, is named 942103-2090_SL.txt and is 4,796 bytes in size.

### FIELD AND BACKGROUND

The present disclosure relates generally to IKK inhibitor that interfere with the IKK-NF-κB signaling pathways, and method of use thereof, for preventing and/or treating inflammation in various diseases and/or numerous inflammatory disease syndromes, including but not limited to, inflammation-induced cancers.

Mobilization of transcription factor NF-κB to the nucleus in response to diverse pro-inflammatory stimuli requires phosphorylation of activation loop serines 177 and 181 within the IKK2/IKKβ subunit of the IκB Kinase (IKK) complex (1). The IKK complex also contains catalytic IKK1/IKKα and accessory NEMO/IKKγ subunits (hereafter referred to as IKK2, IKK1, and NEMO) (2-4). Upon activation, the catalytic IKK2 subunit directs site-specific phosphorylation of the IκBα inhibitor protein, leading to its ubiquitin-dependent degradation via the 26S proteasome and release of the classical NF-κB p50:RelA heterodimer, which migrates into the nucleus to direct response gene expression (5,6). As illustrated by gene knockout studies, the NEMO subunit of the IKK complex is required for induction of NF-κB (7,8). Moreover, before the IKK complex had even been identified unambiguously it was shown that induction of IKK catalytic activity from partially purified cell lysates requires both ubiquitin and ATP (9). Ubiquitin assembles into K63- and M1-linked linear polyubiquitin chains in response to early NF-κB signaling events (10,11). Linear polyubiquitin chains associate both covalently and noncovalently with NEMO, however the noncovalent interaction has been proven to be sufficient for induction of NF-κB transcriptional activity through the canonical signaling pathway (12,13).

Three-dimensional structures of free IKK2 and IKK1 have revealed that they adopt similar structural folds (14-17). Both catalytic domain-containing IKK subunits assemble in solution as homodimers. Interestingly, both IKK2 and IKK1 exhibit a strong propensity for higher degree oligomerization through ordered self-association, although the precise nature of the oligomerization differs significantly between the two proteins (15,17). Despite their 50% amino acid sequence identity and 80% sequence homology, IKK2 and IKK1 rely upon unique surface exposed regions to mediate different higher order assemblies in order to render their activation loops accessible for trans phosphorylation. In light of these observations, we previously proposed a model for induction of IKK catalytic potential via activation loop phosphorylation as a consequence of stabilizing catalytic IKK subunit dimers in their “open” conformation (4,15). Under such a mechanism, it is unclear whether the necessary role of NEMO is that of an adaptor that simply co-localizes catalytic subunits with polyubiquitin chains to sites where homo-oligomerization can promote activation loop phosphorylation or if NEMO:polyubiquitin complexes play a more direct role in facilitating IKK2 subunit phosphorylation and consequent catalytic activity.

### SUMMARY

The present disclosure provides compositions, and method of use thereof, to regulate canonical nuclear fact (NF)-κB signaling, particularly via interaction between NEMO/IKKγ and IKK2/IKKβ, so as to act as effective inhibitors of IKK2 activation to prevent and/or treat inflammations in various diseases and/or numerous inflammatory disease syndromes including inflammation-induced cancers.

In certain embodiments, the present disclosure provides that, upon noncovalent binding to linear polyubiquitin, NEMO directly promotes activation loop phosphorylation of the catalytic IKK2 subunit. A second interaction between NEMO and IKK2 was identified that is dependent upon NEMO binding to linear polyubiquitin. This newly identified NEMO:IKK2 interaction interface was mapped to a stretch of six conserved amino acids immediately N-terminal to the Zn-finger domain at the C-terminus of human NEMO and an exposed region of the IKK2 scaffold-dimerization domain (SDD) proximal to its kinase domain (KD) and ubiquitin-like domain (ULD). In certain embodiments, the six conserved amino acids are QRRSPP (SEQ ID NO: 1), corresponding to amino acid residues 384-389 of human NEMO.

A peptide NEMO^{ActPep} comprising the six conserved amino acids is also provided. The present disclosure provides that this peptide NEMO^{ActPep} mediates the linear polyubiquitin-dependent second interaction between NEMO and IKK2. In certain embodiments, the present disclosure provides that this peptide NEMO^{ActPep} serves to inhibit trans phosphorylation of the IKK2 subunit in vitro and blocks canonical NF-κB signaling in cell culture. Therefore, this peptide NEMO^{ActPep} functions as a highly specific and effective inhibitor of the IKK2 activation pathway. The present disclosure further provides that this peptide NEMO^{ActPep} does not block MAP kinase phosphorylation or IKK1 via the non-canonical NF-κB activation pathway.

Accordingly, disclosed herein is a method of treating and/or preventing inflammation in various disease, by targeting the NEMO:IKK2 interaction interface comprising the six conserved amino acids of QRRSPP (SEQ ID NO: 1), corresponding to amino acid residues 384-389 of human NEMO, to effectively inhibit IKK2 activation pathway, but does not block MAP kinase phosphorylation or IKK1 via the non-canonical NF-κB activation pathway. In certain embodiments, provided herein is a method of treating and/or preventing inflammation in a patient in need thereof, comprising administering to the patient a therapeutically effective amount of a composition comprising a peptide derived from the second interaction region of NEMO/IKKγ that effectively inhibit IKK2 activation. In certain embodiments, the peptide comprises the peptide NEMO^{ActPep} comprising the six conserved amino acids of QRRSPP (SEQ ID NO: 1), corresponding to amino acid residues 384-389 of human NEMO. In certain other embodiments, the composition comprising a small molecule and/or a compound that derives from the peptide NEMO^{ActPep} comprising the six conserved amino acids of QRRSPP (SEQ ID NO: 1), corresponding to amino acid residues 384-389 of human NEMO. Such small molecules and/or compounds can be derived via peptidomimetic chemical approaches or any other suitable conversional approaches.

In the methods provided herein, the composition that targets the NEMO:IKK2 interaction interface can be a single and/or a combination with at least another IKK2 inhibitor. In certain embodiments, the composition of the present disclosure can be administered in a single composition, or separately in more than one composition. Accordingly, also provided herein is a composition comprising a therapeutically effective amount of single and/or a combination of at least one peptide of the present disclosure, and/or small molecule compounds derived therefrom, that targets the NEMO:IKK2 interaction interface and functions as an effective and specific IKK2 inhibitor.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIGS. 1A-1C. NEMO primes IKK2 for trans phosphorylation on its activation loop in the presence of linear polyubiquitin. FIG. 1A. Schematics of domains for human IKK1/IKKα, IKK2/IKKβ, and NEMO/IKKγ protein subunits. IKK1 and IKK2 both contain a well defined kinase domain (KD) as well as a ubiquitin-like domain (ULD), a scaffold dimerization domain (SDD), and a NEMO binding domain (NBD). Structurally and/or functionally defined domains of NEMO include a kinase binding domain (KBD), intervening domain (IVD), coiled-coil domains 1 and 2 (CC1 and CC2), and a zinc finger domain (ZF). FIG. 1B. Alternative possible modes of NEMO and polyubiquitin control over IKK2 subunit phosphorylation within the canonical NF-κB signaling pathway. In the first (depicted symbolically on the left fork labeled “1”), polyubiquitin chains generated as a result of pro-inflammatory cytokine engagement with receptors serve to localize IKK subunits on account of their affinity for NEMO adaptor proteins. A second possibility (right fork labeled “2”) is that polyubiquitin binding enables NEMO to directly influence the structural conformation of IKK2. In either case, the result is stabilization of the IKK complex in a conformation that permits trans phosphorylation of the IKK2 subunit activation loop and IKK activity. FIG. 1C. Western blot analysis of in vitro trans phosphorylation of a catalytically inactive form of IKK2 (K44M) by a constitutively active IKK2 (11-669EE). Inclusion of NEMO and linear tetraubiquitin (Ub4) improves efficiency of phosphorylation (lanes 7-9) relative to either Ub4 (lanes 5 and 6) or NEMO (lanes 10-12) alone.

FIGS. 2A-2D. NEMO mediates a secondary interaction with IKK2 upon interaction with linear polyubiquitin. FIG. 2A. Western analysis of in vitro GST-pull-down with purified recombinant IKK2 and NEMO. Primary antibodies are listed on the left of each panel and the presence of each protein is indicated above each lane. Full length NEMO binds full length IKK2 (lanes 7 and 8) while removal of the NBD results in NEMO:IKK2 binding that is dependent upon the presence of linear polyubiquitin (Ub4) (lanes 9-12). FIG. 2B. The C-terminal CC2-ZF (NEMO²⁴¹⁻⁴¹⁹) is sufficient to bind IKK2 in the presence of Ub4 (lanes 9-12) but interacts only very weakly with IKK1 (lanes 7 and 8). FIG. 2C. Removal of the C-terminal ZF (NEMO²⁴¹⁻³⁹⁰) does not affect Ub4-dependent binding to IKK2 (lanes 9 and 10) while further deletion of NEMO residues 384-390 (NEMO²⁴¹⁻³⁸³) disrupts the interaction (lanes 3-8). FIG. 2D. Mutation of NEMO residues 384-389 (lanes 5 and 6) or 384-386 (lanes 3 and 4) is sufficient to disrupt the Ub4-dependent secondary binding of NEMO to IKK2. Asterisks in the bottom panels of FIGS. 2A, 2C, and 2D indicate proteins from the GST-NEMO preparations that, on account of the relatively high amount of GST-NEMO required to pull-down IKK2 via secondary interaction, react nonspecifically with the anti-His primary antibody.

FIGS. 3A-3C. NEMO residues 384-389 are required for full IKK2 activation in cells and the peptide in isolation competitively inhibits secondary site binding. FIG. 3A. Western blot analysis of lysates from HEK293T cells transfected with full length human HA-IKK2 and full length human Myc-NEMO of either native sequence or with residues 384-386 (3G) or 384-389 (6G) mutated to glycines. IKK2 activation loop hyperphosphorylation is observed when both IKK2 and NEMO are overexpressed (top panel, lane 3). Mutant NEMO proteins do not support robust IKK2 activation loop hyperphosphorylation (top panel, lanes 4 and 5). FIG. 3B. Amino acid sequences for peptides consisting of HIV-1 TAT fused flexibly to human NEMO amino acids 375-391 (TAT-NEMO^{ActPeP}) as well as a variant with residues 384-389 mutated to glycine (TAT-NEMO^{ActPep 6G}). FIG. 3B discloses SEQ ID NOS 2-3, respectively, in order of appearance. FIG. 3C. Western blot analysis of GST-pull-downs (top panel) from HA-IKK2-transfected 293 cell lysates in the presence of linear tetraubiquitin and NEMO^{ActPep} and treated with decreasing (200-25 μM) TAT-NEMO^{ActPep} or TAT-NEMO^{ActPep 6G}. The inputs containing lysates augmented with purified proteins and peptides are shown below.

FIGS. 4A-4D. Identification of a docking site for NEMO^{AdtPep}. FIG. 4A. A ribbon diagram representation of the human IKK2 homodimer with one subunit in grey with semitransparent surface rendered and the other colored by domain. On the right is an individual IKK2 monomer with domains labeled and Ca positions of each mutated residue indicated by a red sphere. FIG. 4B. Western blot analysis of lysates from 293 cells transfected with native and mutant full length human HA-IKK2. Mutation of residues Glu441, Lys442, or Asn445 to alanine weakens the polyubiquitin-dependent association of NEMO with IKK2 (lanes 6-13). FIG. 4C. Western blot analysis of GST-pull-downs (top panel) from native and mutant HA-IKK2-transfected 293 cell lysates in the presence of linear tetraubiquitin and NEMO²⁴¹⁻⁴¹⁹. Inputs containing lysates augmented with purified proteins are shown below. FIG. 4D. A close-up view of the locations of Lys441, Glu442, and Asn445 as well as nearby acidic Asp561, Asp562, Glu564, and Glu565 residues that constitute proposed docking site on IKK2 for NEMO^{ActPep}. IKK2 domains are labeled and the kinase activation loop is depicted/labeled.

FIGS. 5A-5D. NEMO^{ActPep} specifically inhibits canonical NF-κB signaling. FIG. 5A. Autoradiography of electrophoretic mobility shift assay (EMSA) with nuclear extracts from HeLa cells treated with TNF-α after pre-incubation with increasing concentrations (10-50 μM) of either TAT-NEMO^{ActPep} or TAT-NEMO^{ActPep 6G}. TAT-NEMO^{ActPep} disrupts NF-κB-dependent shift of radiolabeled probe (lanes 8-12, upper panel) in a dose-dependent manner while control TAT-NEMO^{ActPep 6G} does not (lanes 3-7). Lower panel shows that binding of the constitutive nuclear transcription factor Oct1 to its probe is not affected by TAT-NEMO^{ActPep}. FIG. 5B. Western blot analysis of cytoplasmic (upper four panels) and nuclear extracts (lower two panels) from TNF-α-treated HeLa cells after pre-incubation with increasing concentrations of either TAT-NEMO^{ActPep} or TAT-NEMO^{ActPep 6G}. IKK2 activation loop phosphorylation and consequent IκBα degradation and NF-κB ReIA subunit nuclear localization are significantly diminished in the presence of TAT-NEMO^{ActPep} (lanes 9-12). FIG. 5C. Western blot analysis indicates no change in the extent of p100 processing in response to activation of the non-canonical NF−κB pathway in HeLa cells with lymphotoxin β receptor (LTβR) after pre-treatment with increasing concentrations of TAT-NEMO^{ActPep} (lanes 3-7) and TAT-NEMO^{ActPep 6G} (lanes 8-12). FIG. 5D. Western blot analyses reveal that pre-incubation with either TAT-NEMO^{ActPep} or TAT-NEMO^{ActPep 6G} has no effect upon levels of MAP kinase phosphorylation in TNF-α-treated HeLa cells.

FIGS. 6A-6D. NEMO^{ActPep} does not display the toxicity profile associated with ATP-competitive IKK2 inhibitor compound MLN120B. FIG. 6A. Autoradiography of EMSA with nuclear extracts from BMDM cells treated with LPS after pre-incubation with 50 μM of either TAT-NEMO^{ActPep} or TAT-NEMO^{ActPep 6G}. TAT-NEMO^{ActPep} disrupts NF-κB-dependent shift of the radiolabeled probe (lane 3) while control TAT-NEMO^{ActPep 6G} does not (lane 4). FIG. 6B. The effect of TAT-NEMO^{ActPep} pre-treatment on relative mRNA levels of select NF-κB target genes as measured by RT-qPCR of BMDM cells induced by LPS. Data were compared by unpaired t-test. *p<0.05; **p<0.01; ***p<0.001. FIGS. 6C-6D. Protein levels of cytokines IL-1β p and TNF-α in the supernatant of cultured BMDM cells pre-incubated with or without 100 μM TAT-NEMO^{ActPep} or MLN120B prior to TNF-α stimulation.

FIG. 7. Schematic representation of IKK2 subunit stabilization in response to linear polyubiquitin binding to NEMO subunits within the IKK complex. The two NEMO subunits of the IKK complex heterotetramer associate via parallel homotypic coiled-coil interactions upon binding of linear polybuiquitin. This alters NEMO structure and dynamics and promotes second site interaction with IKK2 stabilizing the complex in an open conformation that is primed for activation via trans phosphorylation of its activation segment.

FIG. 8A. Coomassie-stained SDS PAGE analysis of purified recombinant proteins employed in in vitro IKK2 trans auto-phosphorylation kinase assay: full length IKK2 (lane 2), IKK2^{K44M} (lanes 3-8), IKK2^{11-669EE} (lane 9), full length NEMO (lane 10) and linear tetraubiquitin (lane 11). FIGS. 8B-8C. Coomassie-stained SDS PAGE (FIG. 8B) and chromatogram of size exclusion chromatography (FIG. 8C) of a mixture of purified full length human NEMO and linear tetraubiquitin (Ub4). The complex elutes as peak 1 and free Ub4 is peak 2.

FIG. 9A. Coomassie-stained SDS PAGE of purified recombinant IKK2 and GST-NEMO protein deletion constructs employed in mapping the secondary binding site on to residues 384-389 of human NEMO. FIGS. 9B-9C. Coomassie-stained SDS PAGE (FIG. 9B) and chromatogram of size exclusion chromatography (FIG. 9C) of a mixture of purified NEMO²⁴¹⁻⁴¹⁹ and linear tetraubiquitin (Ub4). The complex elutes as peak 1 and free Ub4 is peak 2. FIGS. 9D-9F. Coomassie-stained SDS PAGE (FIG. 9D) and chromatograms of size exclusion chromatography of a mixture of purified NEMO²⁵⁰⁻³⁶⁵ and linear tetraubiquitin (Ub4) (FIG. 9E) and free Ub4 (FIG. 9F). The complex elutes as peak 1 and free Ub4 is peak 2. FIG. 9G. Comparison of primary amino acid sequences from the C-terminal ends of mammalian NEMO proteins. The proposed second binding site (labeled) is a conserved sequence of six amino acids corresponding to human NEMO residues 384-389 within a proline-rich region linking the CC2 and ZF (both labeled). Identical residues are present in mouse, bovine, and rat NEMO proteins. The Drosophila IKKγ homolog, which has not been shown conclusively to be required for Drosophila IKKβ activation in response to linear polyubiquitin, lacks this motif.

FIG. 9G discloses SEQ ID NOS 4-8, respectively, in order of appearance. FIG. 10A-10D. Western blots for pull-down experiments in FIGS. 2A-2D. FIGS. 10A-10D correspond to the respective panels in FIGS. 2A-2D. Purified proteins used in the experiment are detected prior to GST-pull-down (an “Inputs”) in lanes −7 through 0. Lanes 1-12 are the same as in FIGS. 2A-2D but the entire blots are shown. Excess GST proteins both in the input and pull-down lanes appear as non-specific “ghost” bands in anti-His blots in panels FIGS. 10B and 10D.

FIG. 11A. A summary table of all mutant IKK2 proteins employed in mapping the second site of linear polyubiqutin-dependent interaction with NEMO including their observed expression levels and degree of activation loop phosphorylation upon co-transfection with NEMO. FIG. 11B. Western blot analysis monitoring expression of native (WT) and mutant human IKK2 proteins in transfected HEK293T cells. FIG. 11C. Western blot analysis monitoring the extent of IKK2 activation loop phosphorylation in HEK293T cells transfected with WT or mutant IKK2 in the absence (−) or presence (+) of co-transfected NEMO. FIG. 11D. Western blot analysis indicates that mutation of aspartic acid residues 383 and 385 to alanine does not interfere with the ability of IKK2 to become fully active when co-transfected with NEMO into HEK293T cells (lanes 31-34).

FIG. 12 A. Densitometry analysis of Ig-κB DNA binding by HeLa cell nuclear lysates as detected by EMSA (FIG. 5A). Data were compared by unpaired t-test. *p<0.05; **p<0.01. FIG. 12B. Western blot analysis of cytoplasmic (upper four panels) and nuclear extracts (lower two panels) from LPS-treated RAW 264.7 cells after pre-incubation with increasing concentrations of either TAT-NEMO^{ActPep} or TAT-NEMO^{ActPep 6G}. FIGS. 12C-12D. Autoradiography (FIG. 12C) of EMSA with nuclear extracts from RAW 264.7 cells treated with LPS after pre-incubation with increasing concentrations (5-55 μM) of either TAT-NEMO^{ActPep} or TAT-NEMO^{ActPep 6G}. Densitometry analysis of Ig-κB DNA binding by RAW 264.7 cell nuclear lysates as detected by EMSA (FIG. 12D). Data were compared by unpaired t-test. *p<0.05; **p<0.01. FIG. 12E. Autoradiography of EMSA with nuclear extracts from MEF cells treated with TNF-α after pre-incubation with increasing concentrations (10-50 μM) of either TAT-NEMO^{ActPep} or TAT-NEMO^{ActPep 6G}.

FIGS. 13A-13B. Gel shift assays of KB DNA binding activity in purified nuclei from HeLa cells induced with either LPS (FIG. 13A) or TNF-α (FIG. 13B). In both experiments, cells were treated with versions of NEMO^{ActPep} and NEMO^{ActPep 6G}lacking the N-terminal HIV-1 TAT cell permeabilization peptide sequence. Rather, the peptides were introduced to cells in combination with the indicated amounts (in μM) of K16ApoE synthetic transport peptide. This shows that the inhibitor effect on NF-κB activity of NEMO^{ActPep} is not reliant upon the HIV-1 TAT peptide. EMSA of Ig-κB DNA binding by HeLa cell nuclear lysates and autodradiography was performed as described in FIGS. 5A-5D.

### DETAILED DESCRIPTION

The present disclosure provides IKK2 specific and effective inhibitors, and method of use thereof, for treating and/or preventing inflammation in various diseases and/or numerous inflammatory disease syndromes, including but not limited to, inflammation-induced cancers.

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

### Definitions

Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications and patents cited in this specification are cited to disclose and describe the methods and/or materials in connection with which the publications are cited. All such publications and patents are herein incorporated by references as if each individual publication or patent were specifically and individually indicated to be incorporated by reference. Such incorporation by reference is expressly limited to the methods and/or materials described in the cited publications and patents and does not extend to any lexicographical definitions from the cited publications and patents. Any lexicographical definition in the publications and patents cited that is not also expressly repeated in the instant application should not be treated as such and should not be read as defining any terms appearing in the accompanying claims. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Aspects of the present disclosure will employ, unless otherwise indicated, techniques of molecular biology, microbiology, organic chemistry, biochemistry, physiology, cell biology, blood vessel biology, and the like, which are within the skill of the art. Such techniques are explained fully in the literature.

Prior to describing the various aspects of the present disclosure, the following definitions are provided and should be used unless otherwise indicated. Additional terms may be defined elsewhere in the present disclosure.

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used herein, the term “and/or” includes any and all combinations of one or more of the associated listed items. Expressions such as “at least one of,” when preceding a list of elements, modify the entire list of elements and do not modify the individual elements of the list.

As used in the specification and the appended claims, the singular forms “a,” “an” and “the” include plural referents unless the context clearly dictates otherwise. Thus, for example, reference to “a therapeutic agent,” “a therapeutic agent that targets the AMPK/caspase-6 axis,” or “a capase-6 inhibitor,” including, but not limited to, two or more such therapeutic agents, therapeutic agents that target the AMPK/caspase-6 axis, or capase-6 inhibitors, including combinations of therapeutic agents, therapeutic agents that target the AMPK/caspase-6 axis, or capase-6 inhibitors, and the like.

Reference to “a/an” chemical compound, therapeutic agent, and pharmaceutical composition each refers to one or more molecules of the chemical compound, therapeutic agent, and pharmaceutical composition rather than being limited to a chemical compound, therapeutic agent, and pharmaceutical composition, the one or more molecules may or may not be identical, so long as they fall under the category of the chemical compound, therapeutic agent, and pharmaceutical composition. Thus, for example, “a” therapeutic agent is interpreted to include one or more molecules of the therapeutic agent, where the therapeutic agent molecules may or may not be identical (e.g., comprising different isotope abundances and/or different degrees of hydration or in equilibrium with different conjugate base or conjugate acid forms).

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

Where a range is expressed, a further aspect includes from the one particular value and/or to the other particular value. Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less' and should be interpreted to include the specific ranges of ‘about x’, ‘about y’, and ‘about z’ as well as the ranges of ‘less than x’, less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y’, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, “about,” “approximately,” “substantially,” and the like, when used in connection with a numerical variable, can generally refers to the value of the variable and to all values of the variable that are within the experimental error (e.g., within the 95% confidence interval for the mean) or within +/−10% of the indicated value, whichever is greater. As used herein, the terms “about,” “approximate,” “at or about,” and “substantially” can mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, the terms “optional” or “optionally” means that the subsequently described event or circumstance can or cannot occur, and that the description includes instances where said event or circumstance occurs and instances where it does not.

As used herein, “administering” can refer to an administration that is oral, topical, intravenous, subcutaneous, transcutaneous, transdermal, intramuscular, intra-joint, parenteral, intra-arteriole, intradermal, intraventricular, intraosseous, intraocular, intracranial, intraperitoneal, intralesional, intranasal, intracardiac, intraarticular, intracavernous, intrathecal, intravireal, intracerebral, and intracerebroventricular, intratympanic, intracochlear, rectal, vaginal, by inhalation, by catheters, stents or via an implanted reservoir or other device that administers, either actively or passively (e.g. by diffusion) a composition the perivascular space and adventitia. For example, a medical device such as a stent can contain a composition or formulation disposed on its surface, which can then dissolve or be otherwise distributed to the surrounding tissue and cells. The term “parenteral” can include subcutaneous, intravenous, intramuscular, intra-articular, intra-synovial, intrasternal, intrathecal, intrahepatic, intralesional, and intracranial injections or infusion techniques. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

As used herein, “therapeutic agent” can refer to any substance, compound, molecule, and the like, which can be biologically active or otherwise can induce a pharmacologic, immunogenic, biologic and/or physiologic effect on a subject to which it is administered to by local and/or systemic action. A therapeutic agent can be a primary active agent, or in other words, the component(s) of a composition to which the whole or part of the effect of the composition is attributed. A therapeutic agent can be a secondary therapeutic agent, or in other words, the component(s) of a composition to which an additional part and/or other effect of the composition is attributed. The term therefore encompasses those compounds or chemicals traditionally regarded as drugs, vaccines, and biopharmaceuticals including molecules such as proteins, peptides, hormones, nucleic acids, gene constructs and the like. Examples of therapeutic agents are described in well-known literature references such as the Merck Index (14th edition), the Physicians' Desk Reference (64th edition), and The Pharmacological Basis of Therapeutics (12th edition), and they include, without limitation, medicaments; vitamins; mineral supplements; substances used for the treatment, prevention, diagnosis, cure or mitigation of a disease or illness; substances that affect the structure or function of the body, or pro-drugs, which become biologically active or more active after they have been placed in a physiological environment. For example, the term “therapeutic agent” includes compounds or compositions for use in all of the major therapeutic areas including, but not limited to, adjuvants; anti-infectives such as antibiotics and antiviral agents; analgesics and analgesic combinations, anorexics, anti-inflammatory agents, anti-epileptics, local and general anesthetics, hypnotics, sedatives, antipsychotic agents, neuroleptic agents, antidepressants, anxiolytics, antagonists, neuron blocking agents, anticholinergic and cholinomimetic agents, antimuscarinic and muscarinic agents, antiadrenergics, antiarrhythmics, antihypertensive agents, hormones, and nutrients, antiarthritics, antiasthmatic agents, anticonvulsants, antihistamines, antinauseants, antineoplastics, antipruritics, antipyretics; antispasmodics, cardiovascular preparations (including calcium channel blockers, beta-blockers, beta-agonists and antiarrythmics), antihypertensives, diuretics, vasodilators; central nervous system stimulants; cough and cold preparations; decongestants; diagnostics; hormones; bone growth stimulants and bone resorption inhibitors; immunosuppressives; muscle relaxants; psychostimulants; sedatives; tranquilizers; proteins, peptides, and fragments thereof (whether naturally occurring, chemically synthesized or recombinantly produced); and nucleic acid molecules (polymeric forms of two or more nucleotides, either ribonucleotides (RNA) or deoxyribonucleotides (DNA) including both double-and single-stranded molecules, gene constructs, expression vectors, antisense molecules and the like), small molecules (e.g., doxorubicin) and other biologically active macromolecules such as, for example, proteins and enzymes. The agent may be a biologically active agent used in medical, including veterinary, applications and in agriculture, such as with plants, as well as other areas. The term therapeutic agent also includes without limitation, medicaments; vitamins; mineral supplements; substances used for the treatment, prevention, diagnosis, cure or mitigation of disease or illness; or substances which affect the structure or function of the body; or pro-drugs, which become biologically active or more active after they have been placed in a predetermined physiological environment.

As used herein, “kit” means a collection of at least two components constituting the kit. Together, the components constitute a functional unit for a given purpose. Individual member components may be physically packaged together or separately. For example, a kit comprising an instruction for using the kit may or may not physically include the instruction with other individual member components. Instead, the instruction can be supplied as a separate member component, either in a paper form or an electronic form which may be supplied on computer readable memory device or downloaded from an internet website, or as recorded presentation.

As used herein, “instruction(s)” means documents describing relevant materials or methodologies pertaining to a kit. These materials may include any combination of the following: background information, list of components and their availability information (purchase information, etc.), brief or detailed protocols for using the kit, trouble-shooting, references, technical support, and any other related documents. Instructions can be supplied with the kit or as a separate member component, either as a paper form or an electronic form which may be supplied on computer readable memory device or downloaded from an internet website, or as recorded presentation. Instructions can comprise one or multiple documents and are meant to include future updates.

As used interchangeably herein, “subject,” “individual,” or “patient” can refer to a vertebrate organism, such as a mammal (e.g. human). “Subject” can also refer to a cell, a population of cells, a tissue, an organ, or an organism, preferably to human and constituents thereof.

As used herein, the terms “treating” and “treatment” can refer generally to obtaining a desired pharmacological and/or physiological effect. The effect can be, but does not necessarily have to be, prophylactic in terms of preventing or partially preventing a disease, symptom or condition thereof. The effect can be therapeutic in terms of a partial or complete cure of a disease, condition, symptom or adverse effect attributed to the disease, disorder, or condition. The term “treatment” as used herein can include any treatment of inflammation associated with any disease in a subject, particularly a human and can include any one or more of the following: (a) preventing the disease from occurring in a subject which may be predisposed to the disease but has not yet been diagnosed as having it; (b) inhibiting the disease, i.e., arresting its development; and (c) relieving the disease, i.e., mitigating or ameliorating the disease and/or its symptoms or conditions. The term “treatment” as used herein can refer to both therapeutic treatment alone, prophylactic treatment alone, or both therapeutic and prophylactic treatment. Those in need of treatment (subjects in need thereof) can include those already with the disorder and/or those in which the disorder is to be prevented. As used herein, the term “treating”, can include inhibiting the disease, disorder or condition, e.g., impeding its progress; and relieving the disease, disorder, or condition, e.g., causing regression of the disease, disorder and/or condition. Treating the disease, disorder, or condition can include ameliorating at least one symptom of the particular disease, disorder, or condition, even if the underlying pathophysiology is not affected, e.g., such as treating the pain of a subject by administration of an analgesic agent even though such agent does not treat the cause of the pain.

As used herein, the term “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors within the knowledge and expertise of the health practitioner and which may be well known in the medical arts. In the case of treating a particular disease or condition, in some instances, the desired response can be inhibiting the progression of the disease or condition. This may involve only slowing the progression of the disease temporarily. However, in other instances, it may be desirable to halt the progression of the disease permanently. This can be monitored by routine diagnostic methods known to one of ordinary skill in the art for any particular disease. The desired response to treatment of the disease or condition also can be delaying the onset or even preventing the onset of the disease or condition.

For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. It is generally preferred that a maximum dose of the pharmacological agents of the invention (alone or in combination with other therapeutic agents) be used, that is, the highest safe dose according to sound medical judgment. It will be understood by those of ordinary skill in the art however, that a patient may insist upon a lower dose or tolerable dose for medical reasons, psychological reasons or for virtually any other reasons.

A response to a therapeutically effective dose of a disclosed compound and/or pharmaceutical composition, for example, can be measured by determining the physiological effects of the treatment or medication, such as the decrease or lack of disease symptoms following administration of the treatment or pharmacological agent. Other assays will be known to one of ordinary skill in the art and can be employed for measuring the level of the response. The amount of a treatment may be varied for example by increasing or decreasing the amount of a disclosed compound and/or pharmaceutical composition, by changing the disclosed compound and/or pharmaceutical composition administered, by changing the route of administration, by changing the dosage timing and so on. Dosage can vary, and can be administered in one or more dose administrations daily, for one or several days. Guidance can be found in the literature for appropriate dosages for given classes of pharmaceutical products.

As used herein, the term “prophylactically effective amount” refers to an amount effective for preventing onset or initiation of a disease or condition.

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

The term “pharmaceutically acceptable” describes a material that is not biologically or otherwise undesirable, i.e., without causing an unacceptable level of undesirable biological effects or interacting in a deleterious manner.

The term “pharmaceutically acceptable salts”, as used herein, means salts of the active principal agents which are prepared with acids or bases that are tolerated by a biological system or tolerated by a subject or tolerated by a biological system and tolerated by a subject when administered in a therapeutically effective amount. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include, but are not limited to; sodium, potassium, calcium, ammonium, organic amino, magnesium salt, lithium salt, strontium salt or a similar salt. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include, but are not limited to; those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like.

The term “pharmaceutically acceptable ester” refers to esters of compounds of the present disclosure which hydrolyze in vivo and include those that break down readily in the human body to leave the parent compound or a salt thereof. Examples of pharmaceutically acceptable, non-toxic esters of the present disclosure include C 1-to-C 6 alkyl esters and C 5-to-C 7 cycloalkyl esters, although C 1-to-C 4 alkyl esters are preferred. Esters of disclosed compounds can be prepared according to conventional methods. Pharmaceutically acceptable esters can be appended onto hydroxy groups by reaction of the compound that contains the hydroxy group with acid and an alkylcarboxylic acid such as acetic acid, or with acid and an arylcarboxylic acid such as benzoic acid. In the case of compounds containing carboxylic acid groups, the pharmaceutically acceptable esters are prepared from compounds containing the carboxylic acid groups by reaction of the compound with base such as triethylamine and an alkyl halide, for example with methyl iodide, benzyl iodide, cyclopentyl iodide or alkyl triflate. They also can be prepared by reaction of the compound with an acid such as hydrochloric acid and an alcohol such as ethanol or methanol.

The term “pharmaceutically acceptable amide” refers to non-toxic amides of the present disclosure derived from ammonia, primary C 1-to-C 6 alkyl amines and secondary C 1-to-C 6 dialkyl amines. In the case of secondary amines, the amine can also be in the form of a 5- or 6-membered heterocycle containing one nitrogen atom. Amides derived from ammonia, C 1-to-C 3 alkyl primary amides and C 1-to-C 2 dialkyl secondary amides are preferred. Amides of disclosed compounds can be prepared according to conventional methods. Pharmaceutically acceptable amides can be prepared from compounds containing primary or secondary amine groups by reaction of the compound that contains the amino group with an alkyl anhydride, aryl anhydride, acyl halide, or aroyl halide. In the case of compounds containing carboxylic acid groups, the pharmaceutically acceptable amides are prepared from compounds containing the carboxylic acid groups by reaction of the compound with base such as triethylamine, a dehydrating agent such as dicyclohexyl carbodiimide or carbonyl diimidazole, and an alkyl amine, dialkylamine, for example with methylamine, diethylamine, and piperidine. They also can be prepared by reaction of the compound with an acid such as sulfuric acid and an alkylcarboxylic acid such as acetic acid, or with acid and an arylcarboxylic acid such as benzoic acid under dehydrating conditions such as with molecular sieves added. The composition can contain a compound of the present disclosure in the form of a pharmaceutically acceptable prodrug.

The term “pharmaceutically acceptable prodrug” or “prodrug” represents those prodrugs of the compounds of the present disclosure which are, within the scope of sound medical judgment, suitable for use in contact with the tissues of humans and lower animals without undue toxicity, irritation, allergic response, and the like, commensurate with a reasonable benefit/risk ratio, and effective for their intended use. Prodrugs of the present disclosure can be rapidly transformed in vivo to a parent compound having a structure of a disclosed compound, for example, by hydrolysis in blood. A thorough discussion is provided in T. Higuchi and V. Stella, Pro-drugs as Novel Delivery Systems, V. 14 of the A.C.S. Symposium Series, and in Edward B. Roche, ed., Bioreversible Carriers in Drug Design, American Pharmaceutical Association and Pergamon Press (1987).

As used herein, the term “derivative” refers to a compound having a structure derived from the structure of a parent compound (e.g., a compound disclosed herein) and whose structure is sufficiently similar to those disclosed herein and based upon that similarity, would be expected by one skilled in the art to exhibit the same or similar activities and utilities as the claimed compounds, or to induce, as a precursor, the same or similar activities and utilities as the claimed compounds. Exemplary derivatives include salts, esters, amides, salts of esters or amides, and N-oxides of a parent compound.

As used herein, nomenclature for compounds, including organic compounds, can be given using common names, IUPAC, IUBMB, or CAS recommendations for nomenclature. When one or more stereochemical features are present, Cahn-Ingold-Prelog rules for stereochemistry can be employed to designate stereochemical priority, E/Z specification, and the like. One of skill in the art can readily ascertain the structure of a compound if given a name, either by systemic reduction of the compound structure using naming conventions, or by commercially available software, such as CHEMDRAW™ (Cambridgesoft Corporation, U.S.A.).

It is understood, that unless otherwise specified, temperatures referred to herein are based on atmospheric pressure (i.e. one atmosphere).

Treatment and/or Prevention Method

Disclosed herein is a method of treating and/or preventing inflammation in various disease in a patient in need thereof, comprising administering to the patient a therapeutically effective amount of an IKK2 specific and effective inhibitor that targets the second interaction interface between NEMO/IKKγ and IKK2/IKKβ. In certain embodiments, the inhibitor is the peptide NEMO^{ActPep} comprising six amino acids of QRRSPP (SEQ ID NO: 1), corresponding to amino acid residues 384-389 of human NEMO. In other embodiments, the inhibitor is a small molecule and/or compound derived from the peptide NEMO^{ActPep} and/or the region of NEMO/IKKγ comprising amino acids immediately N-terminal to the zinc finger domain in human NEMO/IKKγ.

In certain embodiments, provided herein is a method for reducing the level of inflammation. Methods for measuring the extent of inflammation are well known in the art. In one embodiment, the level of inflammation is reduced by about 5% to about 100%. In one embodiment, the level of inflammation is reduced by at least about 5%, at least about 10%, at least about 15%, at least about 20%, at least about 25%, at least about 30%, at least about 35%, at least about 40%, at least about 45%, at least 50%, at least about 55%, at least about 60%, at least about 65%, at least about 70%, at least about 75%, at least about 80%, at least about 85%, at least about 90%, at least about 95%, or about 100% in the subject.

Peptide/Small Molecule/Compound Inhibitor of IKK2 Activation

As disclosed herein, in certain embodiments, the IKK2 inhibitor is the peptide NEMO^{ActPep} comprising six amino acids of QRRSPP (SEQ ID NO: 1), corresponding to amino acid residues 384-389 of human NEMO. In other embodiments, the inhibitor can be a small molecule and/or compound derived from the peptide NEMO^{ActPep} and/or the region of NEMO/IKKγ comprising amino acids immediately N-terminal to the zinc finger domain in human NEMO/IKKγ. The IKK2 inhibitors disclosed herein, are highly effective and targeting IKK2 activation pathway, but do not block MAP kinase phosphorylation or IKK1 via the non-canonical NF-κB activation pathway. Therefore, the IKK2 inhibitors disclosed herein can be developed into drugs with less or no toxicity as compared to other inhibitors.

### Dosing and Administration

While it is possible for an active ingredient to be administered alone, it may be preferable to present them as pharmaceutical formulations or pharmaceutical compositions as described below. The formulations, both for veterinary and for human use, of the disclosure comprise at least one of the active ingredients, together with one or more acceptable carriers therefor and optionally other therapeutic ingredients. The carriers must be “acceptable” in the sense of being compatible with the other ingredients of the formulation and physiologically innocuous to the recipient thereof.

Each of the active ingredients can be formulated with conventional carriers and excipients, which will be selected in accord with ordinary practice. Tablets can contain excipients, glidants, fillers, binders and the like. Aqueous formulations are prepared in sterile form, and when intended for delivery by other than oral administration generally will be isotonic. All formulations will optionally contain excipients such as those set forth in the Handbook of Pharmaceutical Excipients (1986). Excipients include ascorbic acid and other antioxidants, chelating agents such as EDTA, carbohydrates such as dextrin, hydroxyalkylcellulose, hydroxyalkylmethylcellulose, stearic acid and the like. The pH of the formulations ranges from about 3 to about 11, but is ordinarily about 7 to 10. The therapeutically effective amount of active ingredient can be readily determined by a skilled clinician using conventional dose escalation studies. Typically, the active ingredient will be administered in a dose from 0.01 milligrams to 2 grams. In one embodiment, the dosage will be from about 10 milligrams to 450 milligrams. In another embodiment, the dosage will be from about 25 to about 250 milligrams. In another embodiment, the dosage will be about 50 or 100 milligrams. In one embodiment, the dosage will be about 100 milligrams. It is contemplated that the active ingredient may be administered once, twice or three times a day. Also, the active ingredient may be administered once or twice a week, once every two weeks, once every three weeks, once every four weeks, once every five weeks, or once every six weeks.

The pharmaceutical composition for the active ingredient can include those suitable for the foregoing administration routes. The formulations can conveniently be presented in unit dosage form and may be prepared by any of the methods well known in the art of pharmacy. Techniques and formulations generally are found in Remington's Pharmaceutical Sciences (Mack Publishing Co., Easton, Pa.). Such methods include the step of bringing into association the active ingredient with the carrier which constitutes one or more accessory ingredients. In general, the formulations are prepared by uniformly and intimately bringing into association the active ingredient with liquid carriers or finely divided solid carriers or both, and then, if necessary, shaping the product.

Formulations suitable for oral administration can be presented as discrete units such as capsules, cachets or tablets each containing a predetermined amount of the active ingredient; as a powder or granules; as a solution or a suspension in an aqueous or non-aqueous liquid; or as an oil-in-water liquid emulsion or a water-in-oil liquid emulsion. The active ingredient may also be administered as a bolus, electuary or paste. In certain embodiments, the active ingredient may be administered as a subcutaneous injection.

A tablet can be made by compression or molding, optionally with one or more accessory ingredients. Compressed tablets can be prepared by compressing in a suitable machine the active ingredient in a free-flowing form such as a powder or granules, optionally mixed with a binder, lubricant, inert diluent, preservative, or surface active agent. Molded tablets may be made by molding in a suitable machine a mixture of the powdered active ingredient moistened with an inert liquid diluent. The tablets may optionally be coated or scored and optionally are formulated so as to provide slow or controlled release of the active ingredient therefrom.

The active ingredient can be administered by any route appropriate to the condition. Suitable routes include oral, rectal, nasal, topical (including buccal and sublingual), vaginal and parenteral (including subcutaneous, intramuscular, intravenous, intradermal, intrathecal and epidural), and the like. It will be appreciated that the preferred route may vary with for example the condition of the recipient. In certain embodiments, the active ingredients are orally bioavailable and can therefore be dosed orally. In one embodiment, the patient is human.

### Pharmaceutical Compositions

The pharmaceutical compositions of the disclosure provide for an effective amount of an IKK2 inhibitor disclosed herein. In certain embodiments, the IKK2 inhibitor is the peptide NEMO^{ActPep} disclosed herein. In other embodiments, the inhibitor can be a small molecule and/or compound derived from the peptide NEMO^{ActPep} and/or the region of NEMO/IKKγ comprising amino acids immediately N-terminal to the zinc finger domain in human NEMO/IKKγ.

When used for oral use for example, tablets, troches, lozenges, aqueous or oil suspensions, dispersible powders or granules, emulsions, hard or soft capsules, syrups or elixirs may be prepared. Compositions intended for oral use may be prepared according to any method known to the art for the manufacture of pharmaceutical compositions and such compositions may contain one or more agents including sweetening agents, flavoring agents, coloring agents and preserving agents, in order to provide a palatable preparation. Tablets containing the active ingredient in admixture with non-toxic pharmaceutically acceptable excipient which are suitable for manufacture of tablets are acceptable. These excipients may be, for example, inert diluents, such as, for example, calcium or sodium carbonate, lactose, lactose monohydrate, croscarmellose sodium, povidone, calcium or sodium phosphate; granulating and disintegrating agents, such as, for example, maize starch, or alginic acid; binding agents, such as, for example, cellulose, microcrystalline cellulose, starch, gelatin or acacia; and lubricating agents, such as, for example, magnesium stearate, stearic acid or talc. Tablets may be uncoated or may be coated by known techniques including microencapsulation to delay disintegration and adsorption in the gastrointestinal tract and thereby provide a sustained action over a longer period. For example, a time delay material such as, for example, glyceryl monostearate or glyceryl distearate alone or with a wax may be employed.

Formulations for oral use may be also presented as hard gelatin capsules where the active ingredient is mixed with an inert solid diluent, for example calcium phosphate or kaolin, or as soft gelatin capsules wherein the active ingredient is mixed with water or an oil medium, such as, for example, peanut oil, liquid paraffin or olive oil.

Aqueous suspensions of the disclosure contain the active materials in admixture with excipients suitable for the manufacture of aqueous suspensions. Such excipients include a suspending agent, such as, for example, sodium carboxymethylcellulose, methylcellulose, hydroxypropyl methylcelluose, sodium alginate, polyvinylpyrrolidone, gum tragacanth and gum acacia, and dispersing or wetting agents such as, for example, a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethyleneoxycetanol), a condensation product of ethylene oxide with a partial ester derived from a fatty acid and a hexitol anhydride (e.g., polyoxyethylene sorbitan monooleate). The aqueous suspension may also contain one or more preservatives such as, for example, ethyl or n-propyl p-hydroxy-benzoate, one or more coloring agents, one or more flavoring agents and one or more sweetening agents, such as, for example, sucrose or saccharin.

Oil suspensions may be formulated by suspending the active ingredient in a vegetable oil, such as, for example, arachis oil, olive oil, sesame oil or coconut oil, or in a mineral oil such as, for example, liquid paraffin. The oral suspensions may contain a thickening agent, such as, for example, beeswax, hard paraffin or cetyl alcohol. Sweetening agents, such as, for example, those set forth above, and flavoring agents may be added to provide a palatable oral preparation. These compositions may be preserved by the addition of an antioxidant such as, for example, ascorbic acid.

Dispersible powders and granules of the disclosure suitable for preparation of an aqueous suspension by the addition of water provide the active ingredient in admixture with a dispersing or wetting agent, a suspending agent, and one or more preservatives. Suitable dispersing or wetting agents and suspending agents are exemplified by those disclosed above. Additional excipients, for example sweetening, flavoring and coloring agents, may also be present.

The pharmaceutical compositions of the disclosure may also be in the form of oil-in-water emulsions. The oily phase may be a vegetable oil, such as, for example, olive oil or arachis oil, a mineral oil, such as, for example, liquid paraffin, or a mixture of these. Suitable emulsifying agents include naturally-occurring gums, such as, for example, gum acacia and gum tragacanth, naturally occurring phosphatides, such as, for example, soybean lecithin, esters or partial esters derived from fatty acids and hexitol anhydrides, such as, for example, sorbitan monooleate, and condensation products of these partial esters with ethylene oxide, such as, for example, polyoxyethylene sorbitan monooleate. The emulsion may also contain sweetening and flavoring agents. Syrups and elixirs may be formulated with sweetening agents, such as, for example, glycerol, sorbitol or sucrose. Such formulations may also contain a demulcent, a preservative, a flavoring or a coloring agent.

The pharmaceutical compositions of the disclosure may be in the form of a sterile injectable preparation, such as, for example, a sterile injectable aqueous or oleaginous suspension. This suspension may be formulated according to the known art using those suitable dispersing or wetting agents and suspending agents which have been mentioned above. The sterile injectable preparation may also be a sterile injectable solution or suspension in a non-toxic parenterally acceptable diluent or solvent, such as, for example, a solution in 1,3-butane-diol or prepared as a lyophilized powder. Among the acceptable vehicles and solvents that may be employed are water, Ringer's solution and isotonic sodium chloride solution. In addition, sterile fixed oils may conventionally be employed as a solvent or suspending medium. For this purpose any bland fixed oil may be employed including synthetic mono- or diglycerides. In addition, fatty acids such as, for example, oleic acid may likewise be used in the preparation of injectables.

The amount of active ingredient that may be combined with the carrier material to produce a single dosage form will vary depending upon the host treated and the particular mode of administration, such as oral administration or subcutaneous injection. For example, a time-release formulation intended for oral administration to humans may contain approximately 1 to 1000 mg of active material compounded with an appropriate and convenient amount of carrier material which may vary from about 5 to about 95% of the total compositions (weight:weight). The pharmaceutical composition can be prepared to provide easily measurable amounts for administration. For example, an aqueous solution intended for intravenous infusion may contain from about 3 to 500 □g of the active ingredient per milliliter of solution in order that infusion of a suitable volume at a rate of about 30 mL/hr can occur. When formulated for subcutaneous administration, the formulation is typically administered about twice a month over a period of from about two to about four months.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents.

The formulations can be presented in unit-dose or multi-dose containers, for example sealed ampoules and vials, and may be stored in a freeze-dried (lyophilized) condition requiring only the addition of the sterile liquid carrier, for example water for injection, immediately prior to use. Extemporaneous injection solutions and suspensions are prepared from sterile powders, granules and tablets of the kind previously described. Preferred unit dosage formulations are those containing a daily dose or unit daily sub-dose, as herein above recited, or an appropriate fraction thereof, of the active ingredient.

In certain embodiments, the inhibitor of the present disclosure may be formulated in any suitable dosage form for an appropriate administration. In certain embodiments, the methods provided herein comprise administering a pharmaceutical composition comprising the inhibitor of the present disclosure and a pharmaceutically acceptable carrier or excipient. Combination formulations and/or treatment according to the present disclosure comprise the inhibitor of the present disclosure together with one or more pharmaceutically acceptable carriers or excipients and optionally other therapeutic agents, now known or later developed, for treating and/or preventing inflammation. Combination formulations containing the active ingredient may be in any form suitable for the intended method of administration.

Canonical nuclear factor (NF)-κB signaling through the Inhibitor of KB Kinase (IKK) complex requires induction of IKK2/IKKβ subunit catalytic activity via specific phosphorylation within its activation loop. This process is known to be dependent upon the accessory ubiquitin-binding subunit NEMO/IKKγ as well as polyubiquitin chains. However, the mechanism through which polyubiquitin binding serves to promote IKK catalytic activity is unclear.

The present disclosure provides that binding of NEMO/IKKγ to linear polyubiquitin promotes a second interaction between NEMO/IKKγ and IKK2/IKKp, distinct from the well characterized interaction of the NEMO/IKKγ N-terminus to the “NEMO binding domain” at the C-terminus of IKK2/IKKp. The location of this second interaction was mapped to a stretch of roughly six amino acids immediately N-terminal to the zinc finger domain in human NEMO/IKKγ. The present disclosure further provides that amino acid residues within this region of NEMO/IKKγ are necessary for binding to IKK2/IKKβ through this secondary interaction in vitro and for full activation of IKK2/IKKβ in cultured cells. Furthermore, a docking site was identified for this segment of NEMO/IKKγ on IKK2/IKKβ within its scaffold-dimerization domain proximal to the kinase domain-ubiquitin-like domain. The present disclosure further provides that a peptide derived from this region of NEMO/IKKγ is capable of interfering specifically with canonical NF-κB signaling in transfected cells. The in vitro biochemical and cell culture-based experiments disclosed in the present disclosure suggest that, as a consequence of its association with linear polyubiquitin, NEMO/IKKγ plays a direct role in priming IKK2/IKKβ for phosphorylation and that this process can be inhibited to specifically disrupt canonical NF-κB signaling.

It is well established that the IKK complex is the central hub of canonical NF-κB signaling initiated from a wide variety of diverse inducing signals. It has also been shown that IKK2 and NEMO are the essential components of the IKK complex that are required for canonical NF-κB signaling and that K63- and linear/M1-linked polyubiquitin chains act through NEMO as intracellular inducers of IKK2 catalytic activity (36). The present disclosure describes the studies that were motivated by an interest in understanding the mechanism through which NEMO and linear polyubiquitin promote IKK2 activation, which is chemically defined by phosphorylation at two serines (Ser177 and Ser181) present within the activation loop of IKK2. In order to appreciate how changes to IKK complex structure and dynamics are associated with its activation, a brief discussion on the structural and dynamic features of both the IKK2 and NEMO subunits is important.

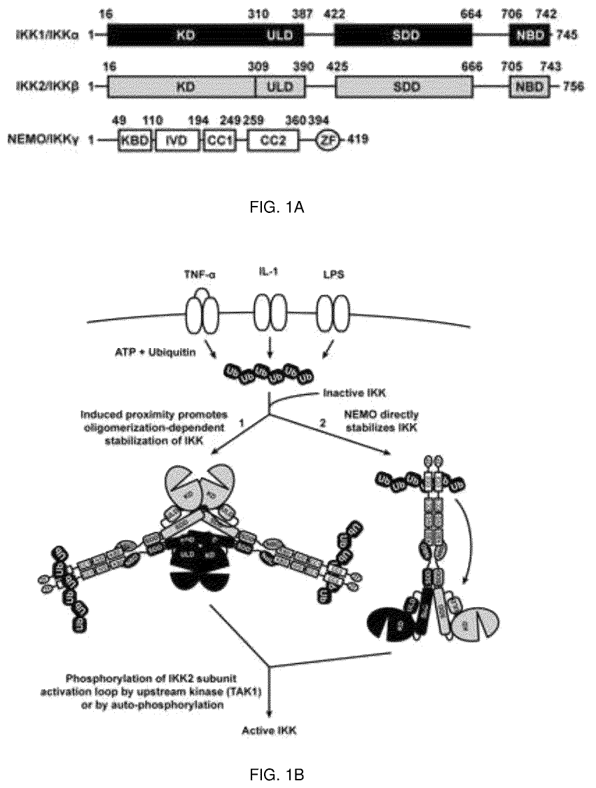

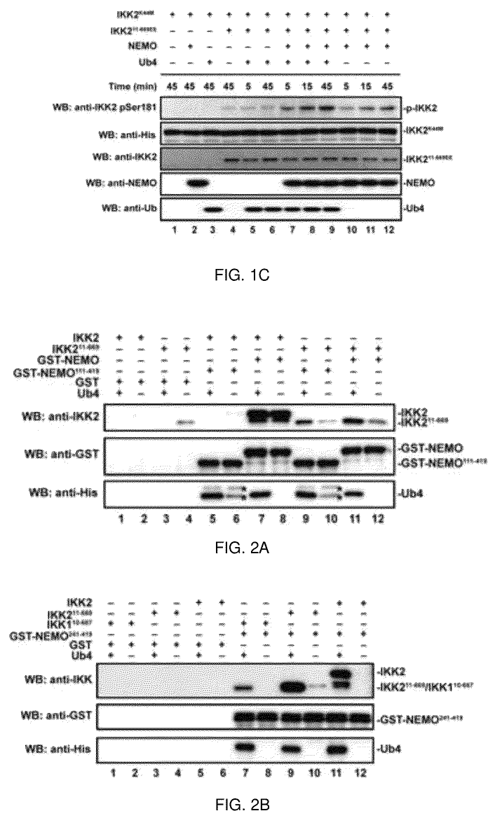

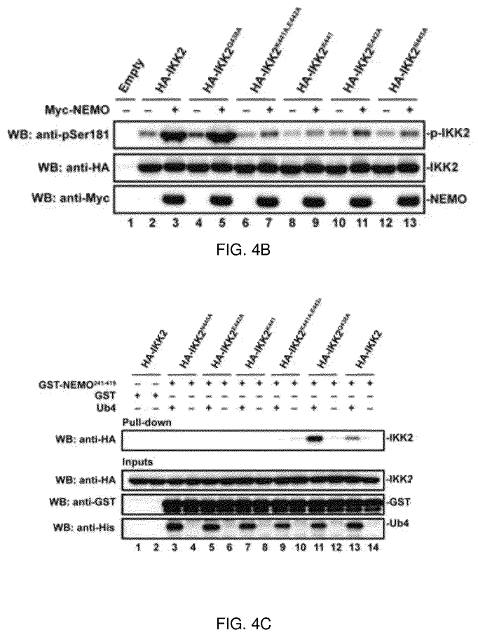

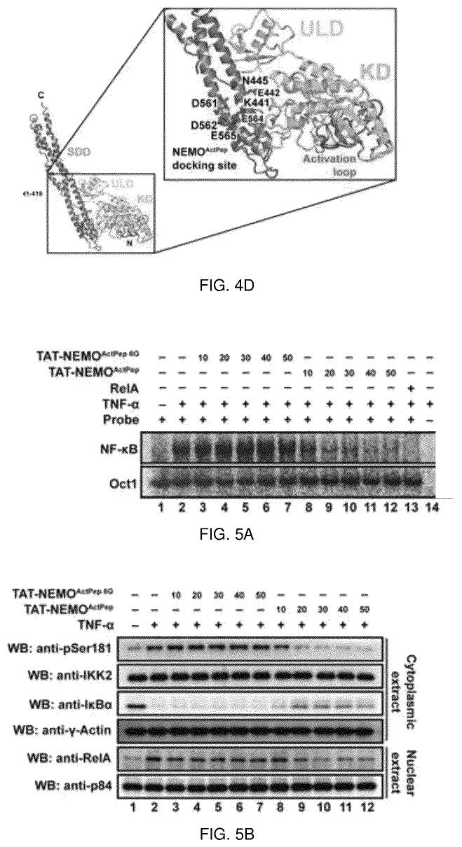

IKK2 has four structurally characterized functional domains. Beginning from the N-terminal end of the protein, these are the KD, ULD, SDD, and NBD (FIG. 1A). The first three adopt folded structures and are connected to one another through non-covalent contacts forming the conserved “IKK-like kinase” core structure (FIG. 4A) (14-17,37,38). The C-terminal NBD is highly dynamic and its main function is to mediate stable interaction with the N-terminal KBD of NEMO, thereby holding the complex together. NEMO in turn is composed of an interrupted series of alpha-helical imperfect coiled-coil (CC) segments (FIG. 1A). Most of these CC segments require binding of accessory factors to stabilize their dimerization. For example, the N-terminal KBD of NEMO binds the IKK2 NBD to form IKK₂:NEMO₂ heterotetramers in solution. The central Ub-binding CC2 domain of NEMO, which spans residues 259-360, can form homodimers on its own, though a NEMO fragment containing both the CC2 and contiguous regions destabilizes CC2 homodimerization suggesting that neighboring portions of NEMO may antagonize dimerization of one another (39,40). Located C-terminal to the CC2 domain of NEMO is the polypeptide sequence QRRSPP (SEQ ID NO: 1) (amino acid residues 384-389) that has been identified as mediating the linear polyubiquitin-dependent second interaction between NEMO and IKK2. The data presented herein suggest that, upon binding to M1-linked linear polyubiquitin, the C-terminal portion of NEMO undergoes structural change exposing the NEMO^{ActPep} sequence for interaction with IKK2 (FIG. 7). Biophysical experimental evidence in support of gross conformational change of NEMO in response to linear polyubiquitin binding in solution has been reported previously (41).

In certain embodiments, the present disclosure provides that the portion of NEMO that contains the NEMO^{ActPep} sequence binds to an exposed “docking site” on the surface of the IKK2 SDD near the KD-ULD. Although the IKK2 docking site for NEMO^{ActPep} is yet to be fully characterized, it is clear from the in vitro mutagenesis and binding data that the QRR tripeptide at NEMO amino acids 384-386 contributes significantly to the overall binding energy of the interaction. Therefore, the present disclosure describes a mechanism for linear polyubiquitin-dependent activation of IKK2 in which free heterotetrameric IKK complexes, each composed of two catalytic subunits as IKK1:IKK2 heterodimers in association with one NEMO₂ homodimer, move through the cell cytoplasm as complex sensors that remain catalytically inactive on account of their inherent flexibility and dynamic character that restricts access of activating protein kinases to their activation loop serines.

Association of the NEMO subunits with linear polyubiquitin induces coiled-coil homodimer formation through the NEMO CC2 domains, which in turn prompts conformational change and association of the exposed NEMO activation peptide region with its docking site on the IKK2 subunit. This second interaction between NEMO and the catalytic IKK subunits stabilizes the IKK1:IKK2 homodimer in an open conformation that is amenable to activation loop trans phosphorylation and thus promotes IKK2 kinase catalytic activity (FIG. 7). Furthermore, interaction of the region of NEMO bearing the NEMO^{ActPep} sequence with the proximal SDD of IKK2 places the C-terminal zinc finger domain of NEMO, which has been shown previously to interact with the substrate IκBα protein, within close proximity of the portion of IKK2 that has also been shown to bind IκBα (14,42). It remains to be determined whether this ligand-dependent kinase stabilization is a general mechanism through which NEMO serves as a versatile integrator of diverse cellular signals or if it is a specific consequence of its interaction with linear polyubiquitin.

The present disclosure further provides that an isolated NEMO^{ActPep} peptide can function as an effective inhibitor of IKK2 activation. In certain embodiments, the present disclosure provides that the NEMO^{ActPep} is a highly specific inhibitor of the IKK2 activation pathway. Consistent with its dependence on a unique interaction between NEMO and IKK2, NEMO^{ActPep} does not block MAP kinase phosphorylation or IKK1 via the non-canonical NF-κB activation pathway. As a central process in inflammatory gene expression, IKK2-dependent induction of NF-κB transcriptional activity has been identified as a key contributor to numerous inflammatory disease syndromes including inflammation-induced cancers (43). Therefore, the canonical NF-κB signaling pathway has long been considered an attractive target for drug development. However, several promising small molecule inhibitors could not be developed into drugs due to their toxicity. In those cases, complete abrogation of IKK2 activity via ATP-competitive binding and potential off target effects appeared to be the main cause for toxicity.

The present disclosure provides that, by the novelty of NEMO^{ActPep} and the specificity it displays for canonical NF-κB signaling, the NEMO^{ActPep} could elicit significant off target effects. Since the length of the peptide that confers inhibition is relatively short, its conversion to small molecule via peptidomimetic chemical approaches is a possible route for generation of novel lead compounds that function to inhibit IKK allosterically by interfering with the ability of linear ubiquitin to trigger NEMO-dependent stabilization of catalytic subunit dimers. Identification of new non-ATP competitive small molecule IKK2 inhibitor lead compounds have been reported recently, though it remains to be seen whether any of these functions through disruption of NEMO:IKK2 second site binding (44).

### Example 1

### Experimental Procedures

### Recombinant Plasmid and Baculovirus Preparation

Human IKK2 cDNA (Uniprot Accession ID: 014920) was graciously provided by the laboratory of M. Karin (UC San Diego School of Medicine). Full length IKK2 was amplified by PCR and cloned in pFastBacHTb (Invitrogen) vector within BamHI and Notl sites in frame with an N-terminal hexahistidine-TEV cleavage site tag “hexahistidine” disclosed as SEQ ID NO: 9. Gene fragments corresponding to human IKK2¹¹⁻⁶⁶⁹ and IKK1¹⁰⁻⁶⁶⁷ were amplified by PCR and subcloned into pFastBacHTb and pFastBacHTa, respectively. For site-directed mutagenesis, codons corresponding to S177 and S181 were mutated to E and K44 to M by PCR-based introduction of deoxyolignucleotide primers harboring the mutations according the Q5 site-directed mutagenesis protocol (New England Biolabs). Recombinant baculovirus production, amplification, and titer optimization were carried out in Sf9 insect cell suspensions as previously described (27).

Full length human NEMO (Uniprot Accession ID: Q9Y6K9) and deletion mutants (NEMO²⁵⁰⁻³⁶⁵, NEMO²⁵⁰⁻⁴¹⁹, and NEMO¹¹¹⁻⁴¹⁹) were subcloned individually into the Ndel and BamHI restriction sites of the pET15b vector in frame with an N-terminal hexahistidine tag (SEQ ID NO: 9). Methionine-linked tetra ubiquitin chain was subcloned into the BamHI and Notl restriction sites of the pET24d vector giving rise to an N-terminal hexahistidine tag (SEQ ID NO: 9) followed by TEV protease recognition sequence.

The glutathione-S-transferase (GST)-NEMO fusions were constructed by subcloning the full length cDNA and deletion variants NEMO²⁴¹⁻³⁵⁰, NEMO²⁴¹⁻³⁷⁵, NEMO²⁴¹⁻²⁸³, NEMO²⁴¹⁻³⁹⁰, NEMO²⁴¹⁻⁴¹⁹, and NEMO¹¹¹⁻⁴¹⁹ into the BamHI and Notl sites of pGEX4T-2 (GE Healthcare Life Sciences) in frame with an N-terminal GST tag followed by a TEV protease recognition sequence. Codons corresponding to Q384-R386 or Q384-P389 in the N-terminal GST-fused NEMO²⁴¹⁻⁴¹⁹ backbone were mutated to encode for G by PCR with base changes incorporated in the oligonucleotide primers.

For cellular assays, full length human IKK2 cDNA was subcloned with an N-terminal HA-tag into pRCCMV-HA (Clontech) vector. Several mutants (total 38; 28 single, 9 double, and 1 triple) of human IKK2 in which select residues were changed to A, S, or G were prepared by PCR with base changes incorporated in the primers. Full length human NEMO was cloned as a Myc-tagged version into pCDNA-3.1 (Invitrogen). Residues 384-386 or 384-389 of the NEMO in pcDNA-3.1 were mutated G by PCR with base substitutions incorporated into the oligonucleotide primers.

### Cell Culture and Reagents

HeLa, HEK 293T, and RAW 264.7 cells were obtained from ATCC and MEF cells were a gift from A. Hoffmann (University of California Los Angeles). Glutathione-agarose beads and Ni-NTA-agarose beads were purchased from BioBharati LifeScience Pvt. Ltd. Mouse anti-HA antibody was purchased from BioLegend. Rabbit anti-NEMO, rabbit anti-IKK2, rabbit anti-His, rabbit anti-Ubiquitin, rabbit anti-Myc, rabbit anti-y-Actin, rabbit anti-ERK2 were purchased from BioBharati LifeScience Pvt. Ltd. Mouse anti-p84 was purchased from GeneTex. Mouse anti-GST, rabbit anti-p65/ReIA, and rabbit anti-IκBα were from Santa Cruz Biotechnology. Rabbit anti-p100/p52 was a gift from N. Rice (National Cancer Institute, Frederick, MD). Rabbit anti-phospho-SAPK/JNK (Thr183/Tyr185), rabbit anti-SAPK/JNK, rabbit anti-phospho-p38 MAPK (Thr180/Tyr182), rabbit anti-p38 MAPK, rabbit anti-phospho-p44/42 MAPK (Erk1/2) (Thr202/Tyr204), and rabbit anti-phospho-IKKα/β (Ser177/181) were purchased from Cell Signaling. Horseradish peroxidase-conjugated anti-rabbit and anti-mouse secondary antibodies were from BioBharati LifeScience Pvt. Ltd. as was mouse TNF-α. LPS was purchased from Sigma. Mouse LTβR was purchased from Abcam.

### Peptides

The 17-mer NEMO^{ActPep} oligopeptide and the mutant NEMO^{ActPep 6G} peptide with six G residues, with and without diglycine-linked N-terminal TAT peptides, were synthesized by Bon Opus Biosciences. Peptides were characterized by matrix-assisted laser desorption ionization mass spectrometry and analytical reverse phase high pressure liquid chromatography analysis. Peptides were dissolved in 1×PBS to stocks of between 2 mM and 10 mM.

### Protein Expression and Purification

All His-tagged NEMO proteins were expressed in Rosetta (DE3) E. coli cells (MilliporeSigma). 1 L cultures in LB media with 100 μg/mL ampicillin were grown at 37° C. to OD600 of 0.2 before induction with 0.2 mM isopropyl-β-D-1-thiogalactopyranoside (IPTG) (BioBharati LifeScience Pvt. Ltd.) and stirring at 150 rpm for 22° C. for 16 hours. Cells were harvested by centrifugation at 3,000×g for 10 minutes (Beckman Coulter) and cell pellets were lysed by sonication (VWR Scientific) on ice in 200 mL of lysis buffer (20 mM Tris-HCl pH 8.0, 500 mM NaCl, 10% w/v glycerol, 10 mM imidazole, 0.2% Triton X-100, 1 mM PMSF, and 5 mM β-mercaptoethanol). Lysates were clarified by centrifugation at 15,000 rpm for 45 minutes. Supernatants containing soluble proteins were then applied to a 1 mL Ni NTA-agarose column that was pre-equilibrated with lysis buffer. Bound proteins were washed with 200 mL wash buffer (lysis buffer with 40 mM imidazole) and eluted in 10 mL elution buffer (lysis buffer containing 150 mM NaCl and 250 mM imidazole).

Sf9 insect cells from 1 L suspension cultures were harvested by centrifugation at 3,000×g for 10 minutes at 4° C. and lysed by sonication in 100 mL of lysis buffer (25 mM Tris-HCl, pH 8.0, 200 mM NaCl, 10 mM imidazole, 10% w/v glycerol, 5 mM β-mercaptoethanol). The lysate was clarified by centrifugation twice at 18,000 rpm for 45 minutes at 4° C. Pre-equilibrated Ni NTA-Agarose resin was added at a ratio of 1 mL of resin slurry/liter of lysed cell culture and the mixture was incubated on a rotator at 4° C. for 3 hours. The Ni beads were pelleted at 1,000 rpm for 2 minutes in a swinging bucket centrifuge rotor. Supernatant was carefully decanted and the protein-bound resin was resuspended with wash buffer (lysis buffer containing 30 mM imidazole) and incubated at 4° C. on a rotator for 2 minutes. The Ni beads were pelleted again and decanted (wash 1). This was repeated until the last wash fraction contained 0.01-0.1 mg/mL of protein (Bio-Rad Protein Assay). Elution buffer (lysis buffer containing 250 mM imidazole) was added, and eluted fractions were collected and stored at −80° C.

Recombinant N-terminal GST-tagged NEMO proteins were expressed in Rosetta (DE3) by growing cells to an A600 of 0.2 followed by induction with 0.2 mM isopropyl β-D-1-thiogalactopyranoside (IPTG) for 16 h at 22° C. Cells were lysed in 200 mL lysis buffer (25 mM Tris (pH 8.0), 500 mM NaCl, 0.1% (v/v) Triton-X 100, 10% (v/v) glycerol, 1 mM ethylenediaminetetraacetic acid (EDTA), 1.0 mM PMSF, and 5 mM β-mercaptoethanol) and sonicated. The lysate was clarified by centrifugation at 15,000 rpm for 45 minutes at 4° C. The supernatant was loaded onto a Glutathione-agarose resin column pre-equilibrated with lysis buffer at 4° C. After wash with 200 mL lysis buffer, protein was eluted with elution buffer (25 mM Tris (pH 8.0), 150 mM NaCl, 0.1% (v/v) Triton X-100, 10% (v/v) glycerol, 5 mM β-mercaptoethanol, and 10 mM glutathione). Eluted fractions were collected and stored at −80° C.

### Fractionation by Size Exclusion Chromatography

Purified individual full length His-tagged IKK2, NEMO, and Ub4 proteins as well as NEMO:Ub4 complexes were subjected to gel filtration with a Superose6 Increase 10/300 GL size-exclusion column (GE Healthcare) on an NGC™ Liquid Chromatography System (Bio-Rad). The column was equilibrated and samples run in buffer (25 mM Tris-HCl, pH 8.0, 250 mM NaCl, 2 mM DTT, and 5% glycerol) at a flow rate of 0.2 mL/min at 22° C. Peak fractions were collected and analyzed by Coomassie-stained SDS-PAGE.

### Whole Cell Extract Preparation and Nuclear-Cytoplasmic Fractionation

HEK293T cells were cultured in DMEM supplemented with 10% (v/v) fetal bovine serum (FBS) and 1% (v/v) penicillin/streptomycin/glutamine. At 70-80% confluence, cells were transiently transfected with empty, HA-IKK2WT, or HA-IKK2 mutant plasmids using polyethylenimine (PEI, PolySciences). Cells were harvested 48 hours post-transfection. To prepare whole cell extracts, cells were lysed in RIPA buffer (10 mM HEPES-KOH pH 7.8, 250 mM NaCl, 1 mM EDTA pH 8.0, 0.5% (v/v) NP-40, 0.2% (v/v) Triton X-100, 2 mM DTT, 20 mM β-glycerophosphate, 10 mM NaF, 0.1 mM Na₃VO₄, 1 mM PMSF, and 1×protease inhibitor cocktail) with gentle shaking for 1 hour at 4° C. Lysed cells were centrifuged at 13,000 rpm for 20 minutes at 4° C., and supernatants containing the whole cell protein extracts were quantified by Bio-Rad protein assay.

To prepare nuclear and cytoplasmic protein extracts, HeLa, MEF, and RAW 264.7 cells were lysed in buffer containing 4.3 mM Na₂HPO₄, 1.47 mM KH₂PO₄ pH 7.4, 137 mM NaCl, 2.7 mM KCl, 1 mM DTT, 0.05% (v/v) NP-40, and 1×protease inhibitor cocktail for 10 minutes on ice and spun at 3,000 rpm at 4° C. for 10 minutes. The supernatant containing the cytoplasmic fraction was quantified by Bio-Rad protein assay to determine the total amount of protein. Pellets were resuspended in nuclear extraction buffer (25 mM Tris-HCl pH 7.5, 420 mM NaCl, 10% glycerol, 0.2 mM EDTA, 1 mM DTT, 0.5 mM PMSF, 1×protease inhibitor cocktail) once and then subjected to three lysis cycles (freeze at −80° C. and thaw at 37° C.). Finally, samples were centrifuged at 13,000 rpm at 4° C. for 20 minutes, and supernatants containing the soluble nuclear fraction were measured by the Bio-Rad protein assay to determine the total amount of protein. Nuclear and cytoplasmic extracts were aliquoted and kept at −80° C.

### Cell Culture, Stimulation, and Peptide Treatment of Cells

HeLa, MEF, RAW 264.7 cells were cultured in Dulbecco's MEM containing 10% fetal bovine and 2 mM L-glutamine serum supplemented with penicillin and streptomycin, at 37° C. in a humidity incubator with 5% CO₂. RAW 264.7 cells were seeded and allowed to adhere for 24 hours then treated with NEMO^{ActPep} or NEMO^{ActPep 6G} at different concentrations 60 min before the LPS challenge (100 ng/ml). After 2 hours, nuclear and cytoplasmic extracts were collected. HeLa and MEF cells seeded for 24 hours then treated with NEMO^{ActPep} or NEMO^{ActPep 6G} at different concentrations 60 min before the TNF-α challenge (10 ng/ml) for 15 minutes and then nuclear and cytoplasmic extracts were collected. MEF cells were treated with NEMO^{ActPep} or NEMO^{ActPep 6G} at different concentrations 1 hour before the LTβR challenge (300 ng/ml). After 24 hours whole cell extracts were collected and aliquoted and kept at −80° C. for further experiments.

### Western Blot Analysis

Equivalent amounts of protein from cell extracts was separated by SDS-PAGE and transferred to polyvinylidene fluoride (PVDF) membranes (Millipore, Bedford, MA, USA). The membrane was blocked with 5% bovine serum albumin (BSA) for 1 hour at room temperature and then the membrane was incubated with the primary antibodies overnight at 4° C. Antibodies for IKK2, His, HA, Myc, GST, Ubiquitin, IκBα, phospho-IKK2, p65/ReIA, p84, γ-Actin, p100/52, phospho-ERK, phospho-JNK, phospho-p38, ERK, JNK, and p38 were used for detecting multiple specific protein targets. After binding of an appropriate secondary antibody coupled to horseradish peroxidase, the immunoreactive bands were visualized by enhanced chemiluminescence substrate.

In Vitro GST Pull-Down Assay with Purified Recombinant Proteins

Glutathione-agarose beads equilibrated with the binding buffer (25 mM Tris-HCl pH 7.5, 150 mM NaCl, 1 mM EDTA, 0.5% NP40, 0.55% Triton X-100, 5% glycerol, 1 mM DTT). Purified recombinant N-terminal GST-NEMO fusion proteins were mixed with recombinant purified IKK2 proteins in the absence or presence of Ub4. The mixtures were incubated in GST binding buffer on a rotating platform for 1 hour at 4° C. then mixed with glutathione-agarose beads. After 2 hours incubation at 4° C., GST-fusion protein complexes bound to glutathione-agarose beads were washed four times with the binding buffer. Proteins bound to the beads were resuspended in SDS-PAGE loading buffer, resolved by SDS-PAGE, and analyzed by western blot.

In Vitro Pull-Down Assay from Whole Cell Extract

Proteins of HEK293T cells transfected with full length human IKK2 or mutants were extracted using RIPA buffer. After centrifugation at 16,000×g for 20 minutes, the supernatants were adjusted to the binding buffer (containing 1.5 mU Hexokinase) and incubated on a rotating platform for 1 hour at 4° C. These whole-cell extracts were then incubated with E. coli-expressed GST-fusion NEMO in the absence or presence of Ub4 on a rotating platform for 2 hours at 4° C. The beads were centrifuged at 1,000×g for 2 minutes and washed four times in binding buffer. Proteins bound to beads were resuspended in SDS loading buffer, resolved by SDS-PAGE, and analyzed by western blot.

For peptide in vitro pull-down assay, proteins of HEK293T cells expressing full length human IKK2 were extracted using the same lysis buffer described above. Whole cell lysates were mixed with E. coli-expressed GST-NEMO with or without NEMO^{ActPep} or mNEMO^{ActPep} at different concentrations in the absence of presence of Ub4 and incubated on a rotating platform for 2 hours at 4° C. The beads were then washed four times in the binding buffer. Proteins bound to beads were resuspended in SDS-PAGE loading buffer, separated by SDS-PAGE, and analyzed by western blot.

### Cell-Based In Vitro Kinase Activity Assay

HEK293T cells were cultured in DMEM supplemented with 10% (v/v) fetal bovine serum (FBS) and 1% (v/v) penicillin/streptomycin/glutamine. Cells were transiently transfected with empty vector, wild type or mutant HA-IKK2, or co-transfected with Myc-NEMO, 3G, or 6G mutant plasmids using PEI following the manufacturer's protocol. After being transfected for 48 hours, cells were harvested and lysed in RIPA buffer for 1 hour at 4° C. Then, cells were centrifuged at 13,000 rpm for 15 minutes at 4° C., and supernatants containing the whole cell protein extracts were quantified by Bio-rad protein assay to determine. Proteins were added SDS-PAGE loading buffer and heated to 95° C. for 5 minutes. Samples were resolved on 10% SDS-PAGE and analyzed by western blot using antibody against phospho-Ser181 IKK2 with anti-HA and -Myc antibodies were used for loading controls. Protein expression was normalized against native sequence IKK2.

### Electrophoretic Mobility Shift Assay

Electrophoretic mobility shift assay (EMSA) was performed using recombinant full length NF-κB ReIA homodimer as a positive control as previously described (45). Briefly, Ig-κB probe was radiolabeled with ³²P-γ-ATP (6,000 Ci/mmol; 10 μCi/μL) and incubated with nuclear lysates for 20 minutes at room temperature in binding buffer (10 mM Tris-HCl pH 7.5, 10% (v/v) glycerol, 1% (v/v) NP-40, 1 mM EDTA, and 0.1 mg/mL salmon sperm DNA). Samples separated in 4% nondenaturing polyacrylamide gels in TGE buffer (24.8 mM Tris base, 190 mM glycine, and 1 mM EDTA) at 200 V for 1 hour, and the gel was dried. The amount of protein in lysates was quantified by Bio-Rad protein assay. EMSA analysis with quantitative densitometry and signal intensity was quantitated using ImageJ software. All EMSA experiments were performed in triplicate.

### BMDM Cell Culture

With the approval of the UCSD and Veteran Affairs San Diego Institutional Animal Care and Use Committees (IACUC), bone marrow-derived macrophage (BMDM) cells were isolated from 6-8 weeks old C57BL/6J mice (Jackson Laboratory) followed by erythrocyte lysis with ammonium chloride, and then seeded in 12-well plates at a concentration of 1×10⁶ cells/mL. Cultured cells were differentiated to macrophages with culture media containing monocyte-colony stimulating factor (M-CSF; 10 ng/mL). After 7 days of differentiation, BMDM cells were treated with TNF-α (10 ng/mL) or LPS (100 g/mL) with or without TAT-NEMO^{ActPep}, TAT-NEMO^{ActPep}6G, or MLN120B.

Extraction of Total RNA and cDNA Synthesis

BMDM cells were pretreated wth or without 50 μM TAT-NEMO^{ActPep} or TAT-NEMO^{ActPep 6G} then stimulated with or without LPS (100 ng/mL) overnight. Cells in the negative control sample were treated with 1×PBS. The total RNA was extracted using TRIzol Reagent (ThermoFisher) according to the manufacturer's instructions. The yield and purity of RNA were determined by absorbance spectrophotometry and RNA samples with optical density 260/280 ratio of 1.8-2.0 were analyzed. First-strand cDNA was synthesized from 2 μg total RNA using 20 μL of Super Reverse Transcriptase MuLV Kit (BioBharati LifeScience Pvt. Ltd.) with random hexamer oligonucleotide primers and incubation at 42° C. for 3 minutes to protect total RNA from genomic DNA interference. Reverse transcription was performed using Super RT enzyme at 50° C. for 15 minutes. Experiments were performed in triplicate.

### Quantitative RT-PCR

For each reaction, 2 μL cDNA product was combined with SYBR Green (New England Biolabs) in a C1000 Touch thermocycler (Bio-Rad) using murine primers to IL-10, TNF-α, RANTES, p52, MIP-1α, MIP1β, MIP-2, MCP-1, IP-10, iNOS, IL-6, IL-2, A20, IL-1β α, IL-1β, IκBα, FAS, COX-2, and GAPDH. The reaction was performed in triplicate. The amplification conditions were as follows: 44 cycles at 95° C. for 15 seconds, 55° C. for 30 seconds, and 0.5° C. gradient increase from 60 to 95° C. Quantitative mRNA expression data were normalized to the expression levels of GAPDH (dCt=Ct gene of interest−Ct GAPDH) and reported as relative mRNA expression (ddCt=2^{-(dCt sample-dCt control)}) or fold change.

### Quantitation of Cytokines

BMDM cels were pre-incubated with or without 100 μM TAT-NEMO^{ActPep} or MLN120B for 24 hours. Negative control cells were treated with 1×PBS or DMSO at the same concentrations as those present in either TAT-NEMO^{ActPep} or MLN120B, respectively. IL-1β and TNF-α levels were measured from 20 μL supernatant of cultured BMDM cells using U-PLEX mouse cytokine assay kit (Meso Scale Diagnostics) following the manufacturer's protocol. The optical density of samples was measured spectrophotometrically. Experiments were performed in triplicate.

### Statistical Analysis

Statistics were calculated with PRISM 8 (version 8.4.3) software (GraphPad). Data were analyzed using unpaired two-tailed Student t-tests for comparison of 2 groups or 1- or 2-way ANOVA for >2 groups followed by Tukey post hoc analysis if appropriate. All data are presented as mean±SEM. P-value <0.05 was considered statistically significant in all analyses.

### Example 2

### Linear Polyubiquitin and NEMO Prime IKK2 for Trans Phosphorylation In Vitro

NEMO, IKK1, and IKK2 associate noncovalently within the IKK complex under physiological conditions and both NEMO and IKK2 are required for stimulus-dependent response gene expression through the canonical NF-κB signaling pathway (FIGS. 1A & 1B) (18). The mechanism underlying the obligatory role of NEMO in promoting IKK2 catalytic activity is unclear. In light of previous observations that catalytic IKK subunits are prone to spontaneous, ordered oligomerization and that, as a consequence of these stabilizing interactions, IKK can undergo phosphorylation-dependent activation in vitro at even moderately higher than cellular concentrations independent of NEMO, NEMO may play a passive adaptor role. Under this mechanism for oligomerization-dependent trans phosphorylation, which was first proposed by Hacker and Karin in 2006, linear polyubiquitin could serve as an anchoring scaffold to recruit and localize multisubunit IKK complexes to intracellular signaling assemblies (19). Alternatively, it is plausible that NEMO may participate directly in priming IKK2 for activation loop phosphorylation and catalytic activity in response to binding linear polyubiquitin (FIG. 1B). Such a mechanism has been suggested by Rahigi, et al. (20).

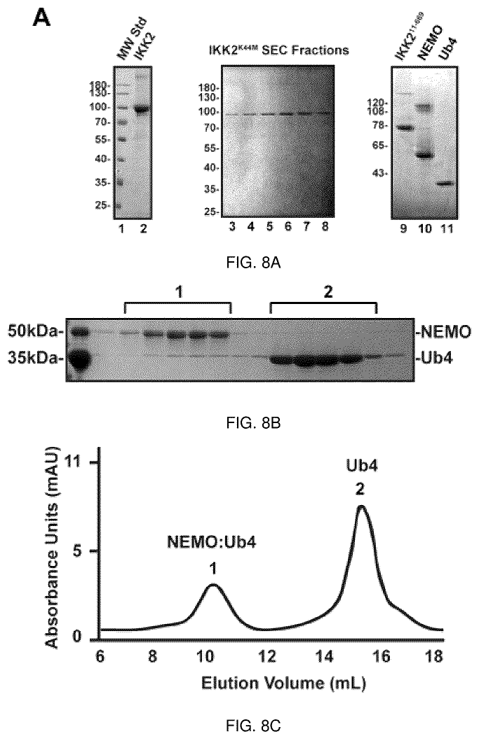

It is examined in vitro with purified recombinant proteins if accessibility of the IKK2 activation loop to protein kinase trans phosphorylation relies upon the binding of linear polyubiquitin and NEMO. A catalytically inactive version of human IKK2 (IKK2^{K44M}) was constructed that contains an otherwise native sequence, unphosphorylated activation loop and, therefore, serves exclusively as a substrate for activation loop trans phosphorylation (FIG. 8A) (15). To this substrate was added a constitutively active version of human IKK2 with both its key activation loop serines (Ser177 and Ser181) mutated to phosphomimetic glutamic acids and its C-terminal NEMO binding domain (NBD) deleted (IKK2^{11-669EE}) at a substrate:enzyme ratio of 2 μg:30 ng. Activation loop phosphorylation was monitored via anti-pSer181 western blot for the substrate IKK2^{K44M}. Linear polyubiquitin chains bind to the coiled-coil 2 (CC2) domain, also known as the “CoZi”, of NEMO (FIG. 1A) (20,21). Therefore, to assess the effect of linear polyubiquitin binding to NEMO on IKK2 activation loop phosphorylation, a recombinant human NEMO was added alone or in combination with linear tetraubiquitin chains (Ub4) generated as four N- to C-terminal covalently-linked ubiquitin moieties (M1-linked). Recombinant Ub4 associates with NEMO in vitro as evidenced by size-exclusion chromatography (FIGS. 8B-8C). Furthermore, Ub4 was deemed as likely sufficient to actuate an effect on IKK2 activation loop phosphorylation since M1-linked diubiquitin is known to bind NEMO in vitro and has been demonstrated to trigger efficient NF-κB activation in cells (22).

It was observed by western blot that the IKK2 activation loop remains largely unphosphorylated in the presence of constitutively active kinase (FIG. 1C). Activation loop phosphorylation increased significantly after reactions were supplemented with NEMO and was enhanced to an even greater extent upon inclusion of both NEMO and Ub4. In the absence of NEMO, Ub4 had no impact on phosphorylation of the IKK2 activation loop. These results suggest that linear polyubiquitin chain binding to NEMO serves to prime the activation loop of IKK2 such that it is amenable to phosphorylation in trans at Ser177 and Ser181.

### Example 3

### Linear Polyubiquitin Induces a Novel Interaction of NEMO and IKK2 Through a “Second” Site

In light of our observation that NEMO efficiently primes the IKK2 activation loop for trans phosphorylation in the presence of linear polyubiquitin, we hypothesized that, in addition to the well characterized high-affinity interaction between the N-terminal segment of NEMO known as the kinase binding domain (KBD) and the C-terminal NEMO binding domain (NBD) of IKK2, polyubiquitin binding might promote additional intermolecular interactions involving other regions of NEMO and IKK2 (23-25). Such polyubiquitin-dependent additional interactions could stabilize IKK2 in its open conformation, which is known to support activation loop phosphorylation in trans either via auto-phosphorylation by IKK2 itself or by other upstream kinases such as TAK1 (26).

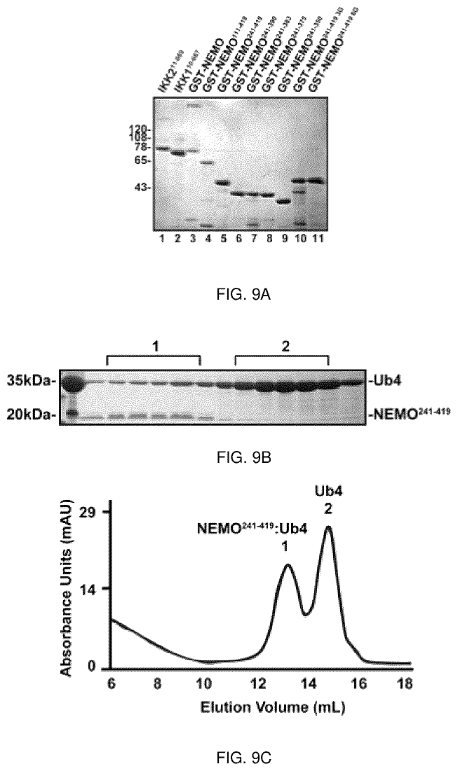

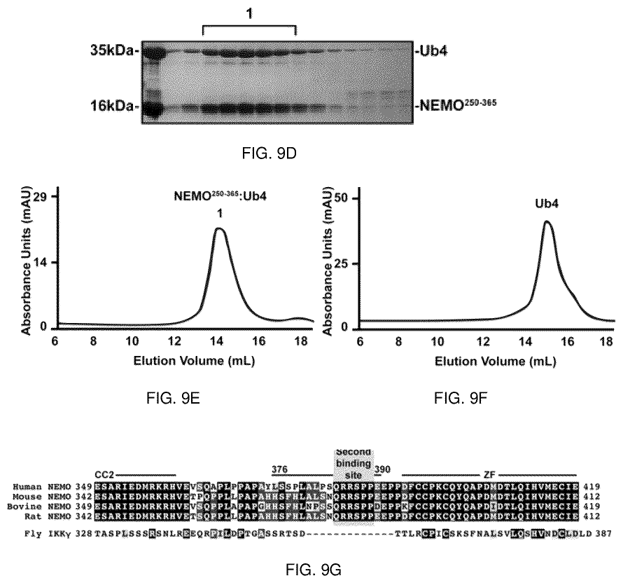

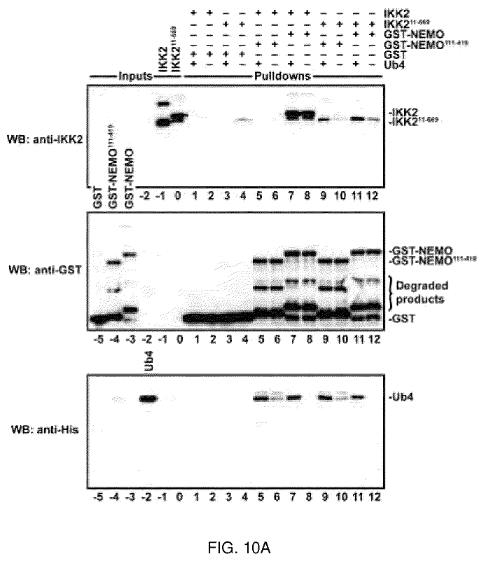

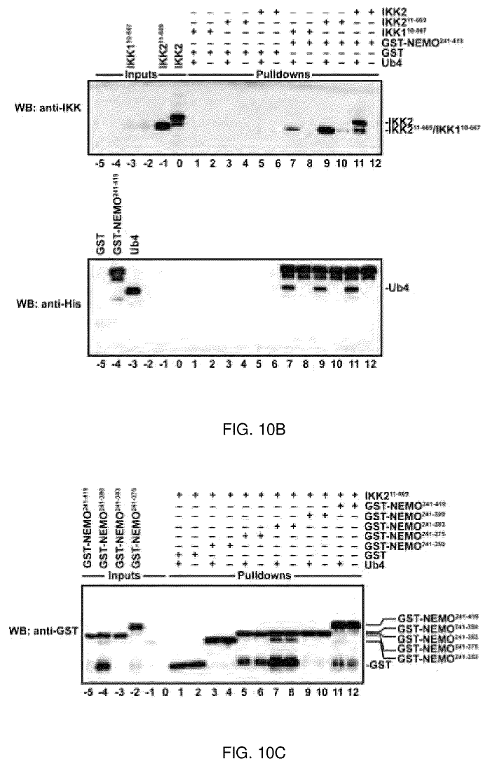

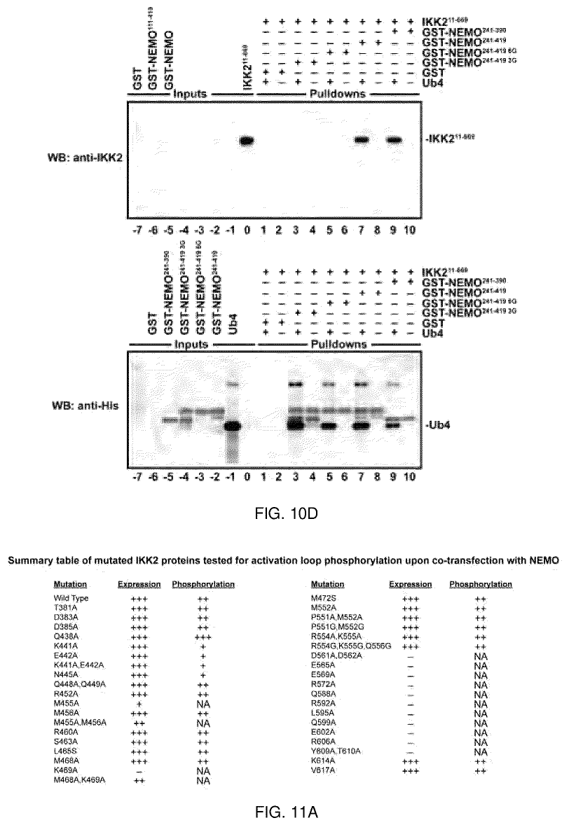

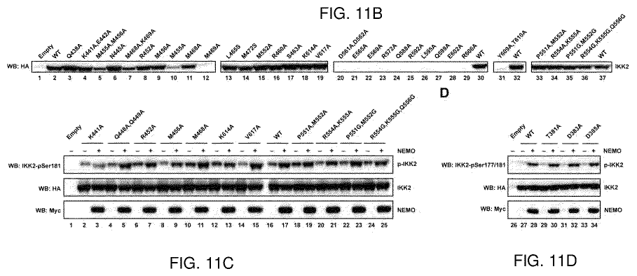

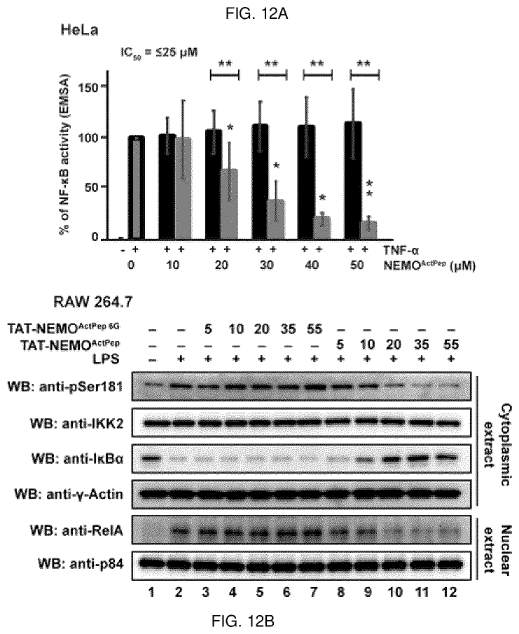

Multiple variants of human NEMO lacking its N-terminal KBD (amino acids 1-110) were produced and the abilities of these NEMO variants to interact with human IKK2 variants lacking their C-terminal NBD (amino acids 670-756) were assessed using in vitro affinity pull-down experiments both in the presence and absence of a linear polyubiquitin (FIGS. 2A-2D). Full length and truncated NEMO were expressed in E. coli and purified to homogeneity as GST-fusion proteins (FIG. 9A). Both full length and truncated (amino acids 111-419) NEMO proteins exhibited association with Ub4, as expected due to their intact CC2 domain (FIG. 2A, lanes 5 and 7; FIGS. 10A-10C). Full length NEMO and full length IKK2 displayed a strong interaction, as expected on account of the well characterized interaction of the IKK2 NBD and NEMO KBD, and the presence of Ub4 did not result in any discernible change to this highly stable complex. Interestingly, the experiment revealed an interaction between IKK2¹¹⁻⁶⁶⁹ (lacking the NBD) and either full length NEMO or NEMO¹¹¹⁻⁴¹⁹ (lacking the KBD) that was enhanced in presence of Ub4 (FIG. 2A; lanes 9-12). This observation is consistent with an additional NEMO:IKK2 interaction that is dependent upon the polyubiquitin binding status of NEMO. Surprisingly, pull-down of full length IKK2 by GST-NEMO¹¹¹⁻⁴¹⁹ in the absence or presence of Ub4 was not observed. This suggests that the native C-terminal NBD of IKK2 interferes with the ability of NEMO to mediate a second interaction in absence of its binding with the NEMO N-terminal KDB.

To define this second interaction more precisely, the region of NEMO that mediates Ub4 binding (20,21) was investigated. GST-NEMO²⁴¹⁻⁴¹⁹, lacking its N-terminal KBD, IVD, and CC1 regions, was first generated and its ability to bind Ub4 was validated both by size-exclusion chromatography and GST pull-down assays (FIG. 1A; FIGS. 9B-9C). Pull-down assays with NEMO²⁴¹⁻⁴¹⁹ and both full length and truncated IKK2 suggest interactions of near equal efficiency as observed with NEMO¹¹¹⁻⁴¹⁹ in the presence of Ub4 but not in its absence (FIG. 2B). The possibility of a similar interaction of NEMO with IKK1 was also investigated. Although an interaction of NEMO with IKK¹¹⁰⁻⁶⁶⁷ in the presence of Ub4 was detected by GST pull-down, it appeared significantly less robust in comparison to that of NEMO and IKK2 (FIG. 2B).

To further pinpoint the region(s) of NEMO involved in the observed second interaction, NEMO²⁴¹⁻³⁹⁰, NEMO²⁴¹⁻³⁸³, NEMO²⁴¹⁻³⁷⁵, and NEMO²⁴¹⁻³⁵⁰ were generated as GST-fusion proteins and consistent with each of the NEMO fragments containing their linear polyubiquitin-binding CC2 region, their ability to bind Ub4 was also confirmed by SEC (FIG. 1A; FIGS. 9D-9F). Pull-down experiments in the presence of Ub4 indicated that both GST-NEMO²⁴¹⁻³⁹⁰ and GST-NEMO²⁴¹⁻⁴¹⁹ are capable of interacting with IKK2¹¹⁻⁶⁶⁹ (FIG. 2C; lanes 9-12). This suggests that the C-terminal Zn-finger domain (amino acids 391-419) of NEMO is not required for the second interaction. However, the observed amount of bound IKK2¹¹⁻⁶⁶⁹ diminished drastically when GST-NEMO²⁴¹⁻³⁸³, GST-NEMO²⁴¹-375, or GST-NEMO²⁴¹⁻³⁵⁰ were employed in the pull-down (FIG. 2C, lanes 3-8). Therefore, a small segment encompassing human NEMO residues 383 to 390 appears to be critical for Ub4-dependent binding of IKK2 through a novel second interaction site.

The amino acid sequences of NEMO from different mammalian species within this region were further analyzed and it was observed a strong conservation of a short segment of polypeptide sequence QRRSPP (SEQ ID NO: 1) spanning residues 384 to 389 (FIG. 9G). To confirm involvement of this small segment in second site binding, two variants of GST-NEMO²⁴¹⁻⁴¹⁹ were generated in which either all six of these residues or only the first three (residues 384-386) were mutated to glycine (NEMO²⁴¹⁻⁴¹⁹⁶G and NEMO^{241-4193G}, respectively). Affinity pull-down experiments with GST-NEMO^{241-419 6G} or GST-NEMO^{241-419 3G} mutant proteins revealed their almost complete absence of interaction with IKK2 in the presence of Ub4 (FIG. 2D). Therefore, it is concluded that NEMO amino acid residues 384-389, which reside immediately N-terminal to the Zn-finger domain, are required for the polyubiquitin-dependent second interaction between NEMO and IKK2 observed in vitro.

### Example 4

### Residues 384-389 of Human NEMO Promote IKK2 Activation Loop Phosphorylation

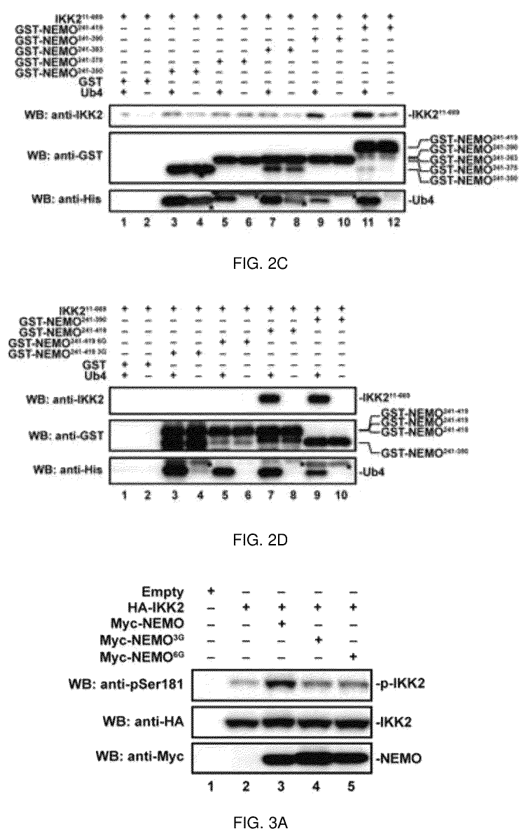

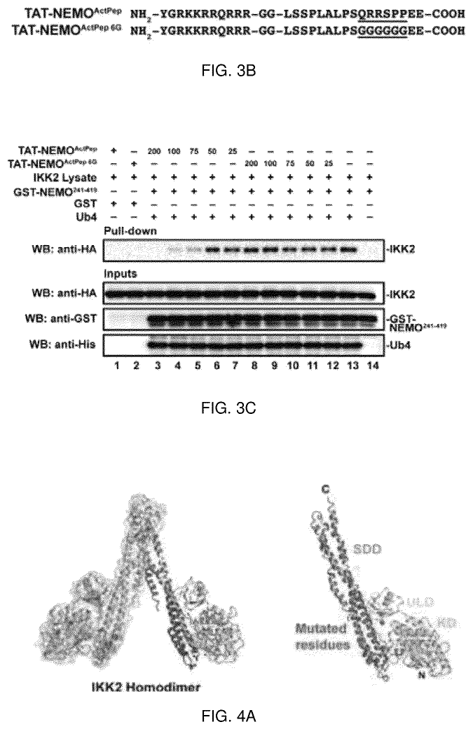

Having established that biding to linear polyubiquitin induces NEMO to interact with IKK2 through a novel, second binding site, the effect of disrupting this interaction was assessed on IKK2 activation in cells. Expression plasmids encoding Myc-tagged full length human NEMO with either residues 384-389 (Myc-NEMO^{6G}) or 384-386 (Myc-NEMO^{3G}) mutated to glycines were generated. These plasmids were transfected in to cultured HEK293T cells and their ability to influence the degree of IKK2 activation was assessed by anti-pSer181 western blot. Earlier studies have revealed that IKK2, when overexpressed in mammalian cells, becomes partially activated, likely as a consequence of its propensity to oligomerize in a manner that promotes its trans phosphorylation (15). The extent of IKK2 activation is further enhanced (hyperactivation) when NEMO is simultaneously overexpressed through co-transfection. Typical hyperactivation of IKK2 was observed when it was co-transfected with full length human Myc-NEMO of native sequence; however, activation was severely diminished with either Myc-NEMO^{6G} or Myc-NEMO^{3G} (FIG. 3A). These results strongly suggest a critical role for the NEMO 384-389 segment in activation of IKK2 within the cell.

### Example 5

A Peptide Derived from NEMO Residues 384-389 Blocks NEMO Second Site Binding to IKK2

Having observed that the region of NEMO encompassing amino acids 384-389 mediates second site binding to IKK2 and influences IKK2 activation in transfected cells, it was further explored if an isolated peptide derived from this region could compete with NEMO for its polyubiquitin-dependent interaction with IKK2. A heptadecapeptide spanning human NEMO amino acid residues 375 and 391, dubbed “NEMO activation peptide” (NEMO^{ActPeP}) was designed and a cell permeable version containing an N-terminal HIV-1 TAT sequence (TAT-NEMO^{ActPeP}) was prepared. A corresponding control peptide with NEMO residues 384-389 altered to glycines (NEMO^{ActPep 6G}) was also generated (FIG. 3B). Pull-down experiments were then performed from extracts of HEK293T cells transfected with plasmids encoding for HA-IKK2 using GST-NEMO²⁴¹⁻⁴¹⁹ alone or in combination with Ub4 and incubated in the absence or presence of increasing concentrations of TAT-NEMO^{ActPep}. As observed with recombinant proteins, GST-NEMO²⁴¹⁻⁴¹⁹ pulled down HA-IKK2 from transfected HEK293T cell lysates only in the presence of Ub4 (FIG. 3C, lanes 13 and 14). a loss of this interaction was observed with increasing concentrations of TAT-NEMO^{AdtPep} (FIG. 3C, lanes 3-7) but not with TAT-NEMO^{ActPep 6G} (FIG. 3C, lanes 8-12). Neither peptide appeared to have any effect upon Ub4 binding to NEMO. These data suggest that the TAT-NEMO^{ActPep} serves to inhibit the polyubiquitin-dependent second interaction between NEMO and IKK2 through competition.

### Example 6

### Identification of a NEMO^{ActPep} Docking Site on IKK2

Truncated IKK2 proteins encompassing only the kinase domain (KD) and ubiquitin-like domain (ULD) are neither regulated properly nor specific toward their substrate IκBα in absence of the IKK2 scaffold-dimerization domain (SDD) (14,27). The SDD resides adjacent to the KD-ULD and mediates subunit dimerization as well as providing flexibility for movement between the open and closed conformations of the IKK2 and IKK1 homodimers (28). Thus, the possibility that the SDD might participate in the observed linear polyubiquitin-dependent second interaction with NEMO was tested through a structure-guided mutagenesis study targeting multiple potential binding residues primarily located within the IKK2 SDD abutting the KD-ULD that has been referred to previously as the “proximal SDD.”

Thirty-eight different residues of IKK2 were altered to generate a total of 38 unique (28 single point mutants, 9 double, and 1 triple) mutant protein constructs (FIG. 4A and FIG. 11A). Upon co-transfection with NEMO into HEK293T cells followed by anti-HA western blot analysis of cellular extracts, several of the mutants showed protein expression defects suggesting that those mutations destabilized protein folding and caused them to be degraded in the cell (FIG. 11B). The activation status of the IKK2 mutants that displayed near normal levels of expression was then assessed by anti-pSer181 western blot. Four of the mutants, Lys441Ala/Glu442Ala, Lys441Ala, Glu442Ala, and Asn445Ala, displayed significantly reduced levels of activation loop phosphorylation compared to wild-type IKK2 indicating that the corresponding residues are critical for IKK2 activation (FIG. 4B and FIG. 11B). Expression levels of these mutants are similar to that of wild-type IKK2 and, as the mutations are located within the exposed surface of the proximal SDD, the observed defects in activation loop phosphorylation are not likely due to global structural perturbations.

To test if these mutants are defective in binding to NEMO through the second site, GST pull-down assays were performed in which E. coli expressed GST-NEMO²⁴¹⁻⁴¹⁹ was mixed with whole cell extracts of HEK293T cells expressing the IKK2 mutant proteins from transfected plasmids, both in the presence or absence of Ub4. Mutation of residues Lys441, Glu442, and Asn445 led to defects in binding (FIG. 4C; FIG. 11C). An IKK2 protein bearing mutation of a nearby residue, Gln438Ala, displayed no defects in phosphorylation of the activation loop and showed even stronger than native-like binding to GST-NEMO²⁴¹⁻⁴¹⁹ in the presence of Ub4, thus serving as a control. The increased activation loop phosphorylation was observed with this mutant protein.

The proximity of Lys441, Glu442, and Asn445 on the surface of the proximal SDD of IKK2 could indicate that these residues constitute a site for the polyubiquitin-dependent second interaction with NEMO. This surface patch the NEMO^{ActPep} “docking site” of IKK2 was dubbed (FIG. 4D). Since NEMO^{ActPep} contains the basic Arg385-Arg386 motif, the likely possibility that an acidic pocket in IKK2 near docking site residues 441, 442, and 445 might also be engaged in NEMO^{ActPep} binding. Two such pockets exist, one within the ULD (Asp383 and Asp385) and the other in the SDD (Asp561, Asp562, Glu564, and Glu565). No defects on IKK2 activation loop phosphorylation in the presence of NEMO were observed upon mutation of either Asp383 or Asp385 to alanine (FIG. 11D). The effect of mutation of IKK2 SDD residues Glu561, Glu562, and Glu564 to alanine could not be assessed since these mutations resulted in loss of IKK2 expression.

### Example 7

### NEMO^{ActPep} Disrupts IKK2 Activation in Cells

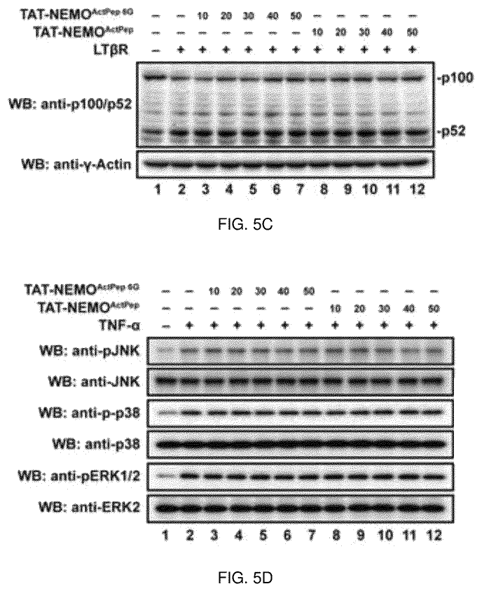

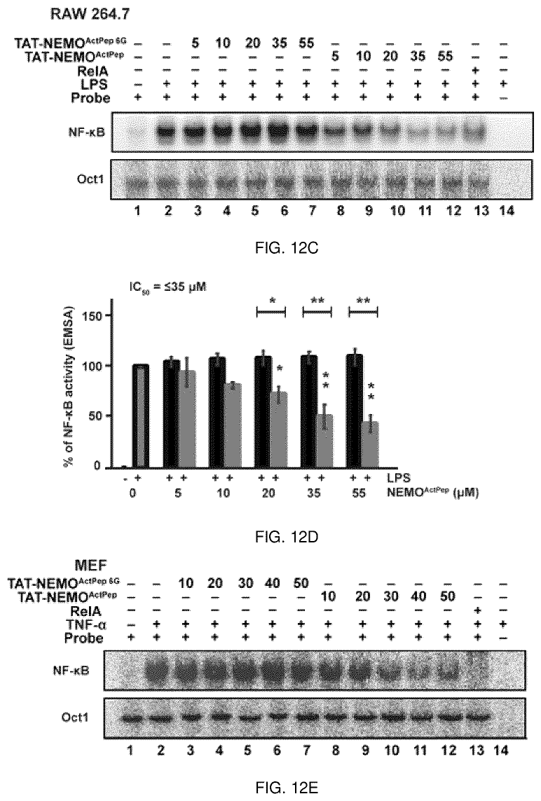

It is intrigued by the ability of such a small segment (residues 384-389) of human NEMO to mediate interaction with and promote activation loop phosphorylation of IKK2. Thus, the potential of a peptide derived from this region to function as a modulator of canonical NF-κB signaling through IKK2 in cells was explored. HeLa S3 cells were pre-treated with either TAT-NEMO^{ActPep} or control TAT-NEMO^{ActPep 6G} for one hour prior to stimulation with 10 ng TNF-α for 15 min. NF-κB activity was measured by EMSA using nuclear extracts and radiolabeled KB DNA from the immunoglobulin kappa light chain gene (Ig-κB) as the probe (FIG. 5A). At 20 μM TAT-NEMO^{ActPep} the amount of shifted probe was significantly reduced and at 40 μM peptide concentration nuclear NF-κB DNA binding activity was nearly abolished (FIGS. 12A-12E). This reduction was not observed in cells treated with the mutant peptide.

The inhibitory effects of TAT-NEMO^{ActPep} were further verified on canonical NF-κB signaling in HeLa cells by assessing IKK2 activation loop phosphorylation, IκBα degradation, and NF-κB ReIA/p65 subunit subcellular localization. Consistent with the observations of decreased nuclear NF-κB DNA binding activity by EMSA, treatment with TAT-NEMO^{ActPep} blocked IKK2 phosphorylation, IκBα proteolysis, and nuclear translocation of ReIA (FIG. 5B). Treatment with the control peptide showed no such effects.

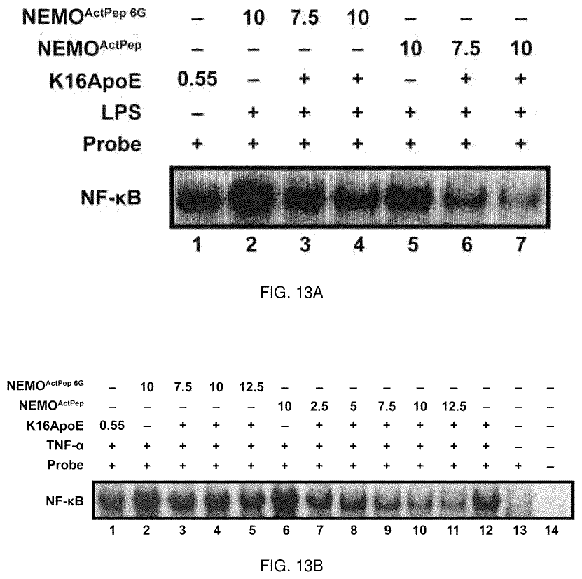

It was then tested if the observed effect is universal across species and cell types. The effect of TAT-NEMO^{ActPep} was tested in RAW 264.7 murine macrophage cells treated with 100 ng/mL purified bacterial lipopolysaccharide (LPS) for 2 hours. Again, NF-κB DNA binding and ReIA subunit nuclear localization were both inhibited as were phosphorylation of the IKK2 activation loop and degradation of IκBα. The mutated peptide did not show such effects (FIG. 12B). EMSA analysis of LPS-treated RAW 264.7 cell nuclear lysates revealed inhibition of NF-κB activity after pre-incubation with TAT-NEMO^{ActPep} relative to TAT-NEMO^{ActPep 6G} to a similar degree as that observed in HeLa cells (FIGS. 12C-12D). The effect of TAT-NEMO^{ActPep} on NF-κB DNA binding was also analyzed in MEF cells by EMSA. Again, TAT-NEMO^{ActPep} but not TAT-NEMO^{ActPep 6}G inhibited NF-κB activation in response to TNF-α (FIG. 12E). Similar experiments in which a version of NEMO^{ActPep} lacking the cell penetrating N-terminal HIV-1 TAT sequence was applied together with K16ApoE synthetic transporter peptide yielded similar results, confirming that the TAT peptide is not responsible for the observed ability of NEMO^{ActPep} to competitively interfere with NEMO:IKK2 second site binding and inhibit canonical NF-κB signaling (FIGS. 13A-13B) (29).

It was also tested if TAT-NEMO^{ActPep} has any effect on non-canonical NF-κB signaling. This alternative NF-κB signaling pathway is activated in response to stimulation of specific cytokine receptors and leads to activation of IKK1 and consequent processing of the p100 precursor to the mature NF-κB subunit p52 (30,31). It was not observed any effect of TAT-NEMO^{ActPep} or TAT-NEMO^{ActPep 6}G on processing of p100 in response to engagement of lymphotoxin-β receptor (LTβR), suggesting involvement of TAT-NEMO^{ActPep} specifically in canonical NF-κB signaling (FIG. 5C). To further investigate the possible involvement of TAT-NEMO^{ActPep} in other signaling pathways, the effect of the peptide on MAPK activation was tested by monitoring the phosphorylation status of JNK, Erk2, and p38 upon TNF-α treatment of HeLa cells. As evidenced by western blot with phospho-specific antibodies, each of the three MAP kinases becomes phosphorylated in response to TNF-α treatment, as expected. Neither TAT-NEMO^{ActPep} nor TAT-NEMO^{ActPep 6G} influenced these phosphorylation processes (FIG. 5D). We conclude that TAT-NEMO^{ActPep} specifically inhibits IKK2 among signaling kinases in response to TNF-α treatment of HeLa cells and disrupts canonical signaling through IκBα to NF-κB.

### Example 8

### NEMO^{ActPep} does not Display Cellular Toxicity Common to IKK Inhibitors

The major problem encountered with ATP-competitive small molecule inhibitors of IKK2 is their toxicity (32). One such compound, MLN120B, has been extensively studied in mice and was shown to cause septic shock due to enhanced levels of pro-IL-1 processing and to promote neutrophilia (33,34). This compound has also been observed to block B- and T-cell proliferation (35). These observations have contributed to the belief that complete inhibition of IKK2 catalytic activity via selective, high affinity ATP-competitive drugs is not a beneficial therapeutic strategy.

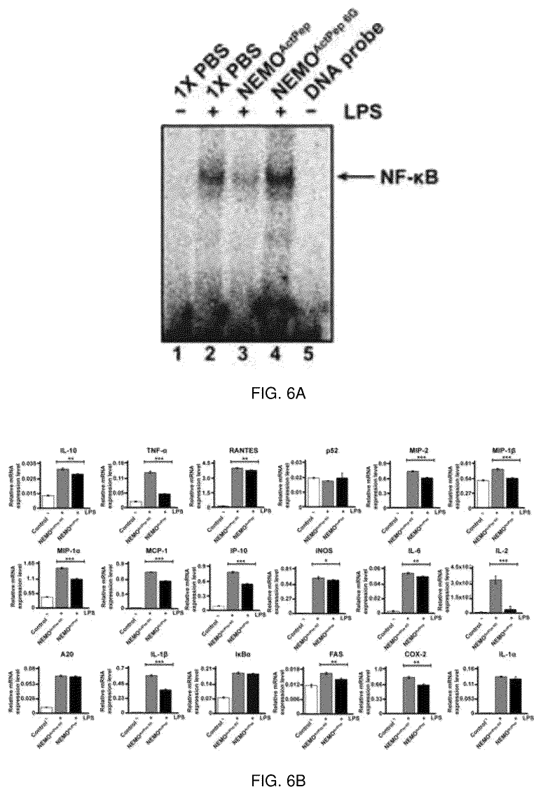

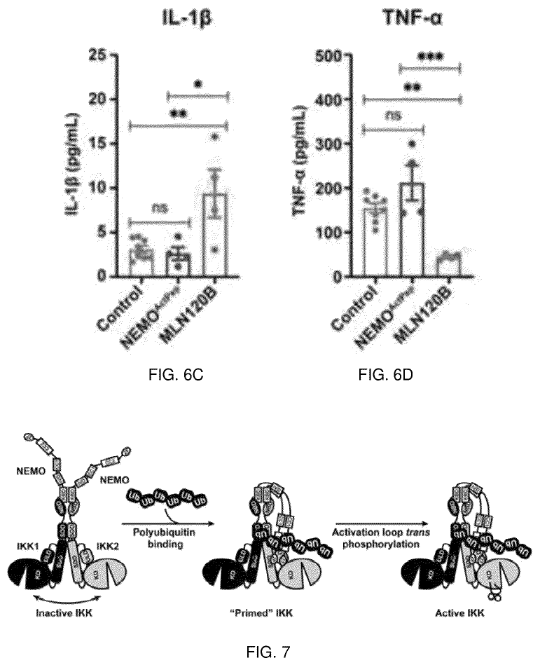

The toxicity of NEMO^{ActPep} in comparison to ATP-competitive inhibitors were tested ex vivo using bone marrow-derived macrophage (BMDM) cells, a murine primary cell line. Consistent with our previous observations, EMSA revealed inhibition of NF-κB activation in response to LPS by pre-treatment with TAT-NEMO^{ActPep} but not TAT-NEMO^{ActPep 6G} (FIG. 6A). mRNA transcript levels were analyzed by RT-qPCR in LPS-induced BMDM cells and it was observed that NEMO^{ActPep} treatment results in significant inhibition of some, but not all, NF-κB-regulated genes (FIG. 6B). This unique pattern of NF-κB regulated gene expression could be due to low level activity of residual nuclear NF-κB, which is sufficient to activate genes with strong KB sites and/or through the actions of other, compensatory transcription factors such as AP1. These observations strongly suggest that NEMO^{ActPep} disrupts NF-κB signaling to a milder extent and against a select subset of NF-κB response genes.

Further, the potentially toxic effects of a high concentration (100 μM) dose of NEMO^{ActPep} were tested on IL-1β production in BMDMs without stimulating the cells. For comparison, BMDM cells were treated with 100 μM MLN120B. ELISA analysis revealed that NEMO^{ActPep} failed to induce production of IL-1β. As reported, however, MLN120B treatment significantly induced production of mature IL-1β in these cells (FIG. 6C, left panel). The peptide also had no effect on TNF-α expression, whereas MLN120B reduced basal levels of TNF-α (FIG. 6C, right panel). These results suggest that disruption of canonical NF-κB signaling with NEMO^{ActPep} does not promote toxic levels of IL-1β as opposed to one well studied ATP-competitive inhibitor.

### REFERENCES

1. Delhase, M., Hayakawa, M., Chen, Y., and Karin, M. (1999) Positive and negative regulation of IκB kinase activity through IKKβ subunit phosphorylation. Science 284, 309-313
2. Karin, M., and Ben-Neriah, Y. (2000) Phosphorylation meets ubiquitination: the control of NF-κB activity. Annu. Rev. Immunol. 18, 621-663
3. Isräel, A. (2010) The IKK complex, a central regulator of NF-κB activation. Cold Spring Harb. Perspect. Biol. 2, a0001 58
4. Mulero, M. C., Huxford, T., and Ghosh, G. (2019) NF-κB, IκB, and IKK: Integral components of immune system signaling. Adv. Exp. Med. Biol. 1172, 207-226
5. Hayden, M. S., and Ghosh, S. (2008) Shared principles in NF-κB signaling. Cell 132, 344-362
6. Huxford, T., Hoffmann, A., and Ghosh, G. (2011) Understanding the logic of IκB:NF-κB regulation in structural terms. Curr. Top. Microbiol. Immunol. 349,1-24
7. Schmidt-Supprian, M., Bloch, W., Courtois, G., Addicks, K., Israel, A., Rajewsky, K., and Pasparakis, M. (2000) NEMO/IKKγ-deficient mice model incontinentia pigmenti. Mol. Cell 5, 981-992
8. Rudolph, D., Yeh, W. C., Wakeham, A., Rudolph, B., Nallainathan, D., Potter, J., Elia, A. J., and Mak, T. W. (2000) Severe liver degeneration and lack of NF-κB activation in NEMO/IKKγ-deficient mice. Genes Dev. 14, 854-862
9. Chen, Z. J., Parent, L., and Maniatis, T. (1996) Site-specific phosphorylation of IκBα by a novel ubiquitination-dependent protein kinase activity. Cell 84, 853-862
10. Deng, L., Wang, C., Spencer, E., Yang, L., Braun, A., You, J., Slaughter, C., Pickart, C., and Chen, Z. J. (2000) Activation of the IκB kinase complex by TRAF6 requires a dimeric ubiquitin-conjugating enzyme complex and a unique polyubiquitin chain. Cell 103, 351-361
11. Tokunaga, F., Sakata, S., Saeki, Y., Satomi, Y., Kirisako, T., Kamei, K., Nakagawa, T., Kato, M., Murata, S., Yamaoka, S., Yamamoto, M., Akira, S., Takao, T., Tanaka, K., and Iwai, K. (2009) Involvement of linear polyubiquitylation of NEMO in NF-κB activation. Nat. Cell Biol. 11, 123-132
12. Hadian, K., Griesbach, R. A., Dornauer, S., Wanger, T. M., Nagel, D., Metlitzky, M., Beisker, W., Schmidt-Supprian, M., and Krappmann, D. (2011) NF-κB essential modulator (NEMO) interaction with linear and Lys-63 ubiquitin chains contributes to NF-κB activation. J. Biol. Chem. 286, 26107-26117
13. Chen, J., and Chen, Z. J. (2013) Regulation of NF-κB by ubiquitination. Curr. Opin. Immunol. 25, 4-12
14. Xu, G., Lo, Y. C., Li, Q., Napolitano, G., Wu, X., Jiang, X., Dreano, M., Karin, M., and Wu, H. (2011) Crystal structure of inhibitor of κB kinase β. Nature 472, 325-330
15. Polley, S., Huang, D. B., Hauenstein, A. V., Fusco, A. J., Zhong, X., Vu, D., Schröfelbauer, B., Kim, Y., Hoffmann, A., Verma, I. M., Ghosh, G., and Huxford, T. (2013) A structural basis for IκB kinase 2 activation via oligomerization-dependent trans auto-phosphorylation. PLoS Biol. 11, e1001581
16. Liu, S., Misquitta, Y. R., Olland, A., Johnson, M. A., Kelleher, K. S., Kriz, R., Lin, L. L., Stahl, M., and Mosyak, L. (2013) Crystal structure of a human IκB kinase μ asymmetric dimer. J. Biol. Chem. 288, 22758-22767
17. Polley, S., Passos, D. O., Huang, D. B., Mulero, M. C., Mazumder, A., Biswas, T., Verma, I. M., Lyumkis, D., and Ghosh, G. (2016) Structural basis for the activation of IKK1/α. Cell Rep. 17, 1907-1914
18. Rothwarf, D. M., Zandi, E., Natoli, G., and Karin, M. (1998) IKKγ is an essential regulatory subunit of the IκB kinase complex. Nature 395, 297-300
19. Häcker, H., and Karin, M. (2006) Regulation and function of IKK and IKK-related kinases. Sci. STKE2006, re13
20. Rahighi, S., Ikeda, F., Kawasaki, M., Akutsu, M., Suzuki, N., Kato, R., Kensche, T., Uejima, T., Bloor, S., Komander, D., Randow, F., Wakatsuki, S., and Dikic, I. (2009) Specific recognition of linear ubiquitin chains by NEMO is important for NF-κB activation. Cell 136, 1098-1109
21. Lo, Y. C., Lin, S. C., Rospigliosi, C. C., Conze, D. B., Wu, C. J., Ashwell, J. D., Eliezer, D., and Wu, H. (2009) Structural basis for recognition of diubiquitins by NEMO. Mol. Cell 33, 602-615
22. Kensche, T., Tokunaga, F., Ikeda, F., Goto, E., Iwai, K., and Dikic, I. (2012) Analysis of nuclear factor-KB (NF-κB) essential modulator (NEMO) binding to linear and lysine-linked ubiquitin chains and its role in the activation of NF-κB. J. Biol. Chem. 287, 23626-23634
23. May, M. J., D'Acquisto, F., Madge, L. A., Glockner, J., Pober, J. S., and Ghosh, S. (2000) Selective inhibition of NF-κB activation by a peptide that blocks the interaction of NEMO with the IκB kinase complex. Science 289, 1550-1554
24. May, M. J., Marienfeld, R. B., and Ghosh, S. (2002) Characterization of the IκB-kinase NEMO binding domain. J. Biol. Chem. 277, 45992-46000
25. Rushe, M., Silvian, L., Bixler, S., Chen, L. L., Cheung, A., Bowes, S., Cuervo, H., Berkowitz, S., Zheng, T., Guckian, K., Pellegrini, M., and Lugovskoy, A. (2008) Structure of a NEMO/IKK-associating domain reveals architecture of the interaction site. Structure 16, 798-808
26. Zhang, J., Clark, K., Lawrence, T., Peggie, M. W., and Cohen, P. (2014) An unexpected twist to the activation of IKKp: TAK1 primes IKKβ for activation by autophosphorylation. Biochem. J. 461, 531-537
27. Shaul, J. D., Farina, A., and Huxford, T. (2008) The human IKKP subunit kinase domain displays CK2-like phosphorylation specificity. Biochem. Biophys. Res. Commun. 374, 592-597
28. Hauenstein, A. V., Rogers, W. E., Shaul, J. D., Huang, D. B., Ghosh, G., and Huxford, T. (2014) Probing kinase activation and substrate specificity with an engineered monomeric IKK2. Biochemistry 53, 2064-2073
29. Mahlum, E., Mandal, D., Halder, C., Maran, A., Yaszemski, M. J., Jenkins, R. B., Bolander, M. E., and Sarkar, G. (2007) Engineering a noncarrier to a highly efficient carrier peptide for noncovalently delivering biologically active proteins into human cells. Anal. Biochem. 365, 215-221
29. Sarkar, G., Curran, G. L., Mahlum, E., Decklever, T., Wengenack, T. M., Blahnik, A., Hoesley, B., Lowe, V. J., Poduslo, J. F., and Jenkins, R. B. (2011) A carrier for non-covalent delivery of functional β-galactosidase and antibodies against amyloid plaques and IgM to the brain. PLoS One 6, e28881
30. Senftleben, U., Cao, Y., Xiao, G., Greten, F. R., Krahn, G., Bonizzi, G., Chen, Y., Hu, Y., Fong, A., Sun, S. C., and Karin, M. (2001) Activation by IKKα of a second, evolutionary conserved, NF-κB signaling pathway. Science 293, 1495-1499
31. Xiao, G., Harhaj, E. W., and Sun, S. C. (2001) NF-κB-inducing kinase regulates the processing of NF-κB2 p100. Mol. Cell 7, 401-409
32. Gamble, C., McIntosh, K., Scott, R., Ho, K. H., Plevin, R., and Paul, A. (2012) Inhibitory KB Kinases as targets for pharmacological regulation. Br. J. Pharmacol. 165, 802-819
33. Greten, F. R., Arkan, M. C., Bollrath, J., Hsu, L. C., Goode, J., Miething, C., Goktuna, S. I., Neuenhahn, M., Fierer, J., Paxian, S., Van Rooijen, N., Xu, Y., O'Cain, T., Jaffee, B. B., Busch, D. H., Duyster, J., Schmid, R. M., Eckmann, L., and Karin, M. (2007) NF-κB is a negative regulator of IL-1β secretion as revealed by genetic and pharmacological inhibition of IKKβ. Cell 130, 918-931
34. Lopez-Castejon, G., and Brough, D. (2011) Understanding the mechanism of IL-1β p secretion. Cytokine Growth Factor Rev. 22, 189-195
35. Nagashima, K., Sasseville, V. G., Wen, D., Bielecki, A., Yang, H., Simpson, C., Grant, E., Hepperle, M., Harriman, G., Jaffee, B., Ocain, T., Xu, Y., and Fraser, C. C. (2006) Rapid TNFR1-dependent lymphocyte depletion in vivo with a selective chemical inhibitor of IKKβ. Blood 107, 4266-4273
36. Hu, H., and Sun, S. C. (2016) Ubiquitin signaling in immune responses. Cell Res. 26, 457-483
37. Larabi, A., Devos, J. M., Ng, S. L., Nanao, M. H., Round, A., Maniatis, T., and Panne, D. (2013) Crystal structure and mechanism of activation of TANK-binding kinase 1. Cell Rep. 3, 734-746
38. Tu, D., Zhu, Z., Zhou, A. Y., Yun, C. H., Lee, K. E., Toms, A. V., Li, Y., Dunn, G. P., Chan, E., Thai, T., Yang, S., Ficarro, S. B., Marto, J. A., Jeon, H., Hahn, W. C., Barbie, D. A., and Eck, M. J. (2013) Structure and ubiquitination-dependent activation of TANK-binding kinase 1. Cell Rep. 3, 747-758
39. Shaffer, R., DeMaria, A. M., Kagermazova, L., Liu, Y., Babaei, M., Caban-Penix, S., Cervantes, A., Jehle, S., Makowski, L., Gilmore, T. D., Whitty, A., and Allen, K. N. (2019) A central region of NF-κB essential modulator is required for IKKβ-induced conformational change and for signal propagation. Biochemistry 58, 2906-2920
40. Ko, M. S., Biswas, T., Mulero, M. C., Bobkov, A. A., Ghosh, G., and Huxford, T. (2020) Structurally plastic NEMO and oligomerization prone IKK2 subunits define the behavior of human IKK2:NEMO complexes in solution. Biochim. Biophys. Acta Proteins Proteom. 1868, 140526
41. Hauenstein, A. V., Xu, G., Kabaleeswaran, V., and Wu, H. (2017) Evidence for M1-linked polyubiquitin-mediated conformational change in NEMO. J. Mol. Biol. 429, 3793-3800
42. Schröfelbauer, B., Polley, S., Behar, M., Ghosh, G., and Hoffmann, A. (2012) NEMO ensures signaling specificity of the pleiotropic IKKβ by directing its kinase activity toward IκBα. Mol. Cell 47, 111-121
43. Taniguchi, K., and Karin, M. (2018) NF-κB, inflammation, immunity and cancer: coming of age. Nat. Rev. Immunol. 18, 309-324
44. Napoleon, J. V., Singh, S., Rana, S., Bendjennat, M., Kumar, V., Kizhake, S., Palermo, N. Y., Ouellette, M. M., Huxford, T., and Natarajan, A. (2021) Small molecule binding to inhibitor of nuclear factor KB kinase subunit p in an ATP non-competitive manner. Chem. Commun. 57, 4678-4681
45. Mulero, M. C., Shahabi, S., Ko, M. S., Schiffer, J. M., Huang, D. B., Wang, V. Y., Amaro, R. E., Huxford, T., and Ghosh, G. (2018) Protein cofactors are essential for high-affinity DNA binding by the nuclear factor KB ReIA subunit. Biochemistry 57, 2943-2957

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1. An IKK2 inhibitor that specifically inhibits IKK2 activation by targeting a second interaction interface between NEMO and IKK2 that is dependent upon NEMO binding to linear polyubiquitin, said IKK2 inhibitor does not block MAP kinase phosphorylation or IKK1 via a non-canonical NF-κB activation pathway.

2. The IKK2 inhibitor of claim 1, wherein the second interaction interface comprises amino acids immediately N-terminal to a Zn-finger domain at a C-terminus of NEMO.

3. The IKK2 inhibitor of claim 1, wherein the inhibitor is a peptide derived from the second interaction interface of NEMO.

4. The IKK2 inhibitor of claim 3, wherein the inhibitor is a peptide NEMO^{ActPep} comprising six amino acids QRRSPP (SEQ ID NO: 1) corresponding to amino acid residues 384-289 of NEMO.

5. The IKK2 inhibitor of claim 4, wherein the inhibitor is a small molecule converted from the peptide NEMO^{ActPep}.

6. The IKK2 inhibitor of claim 5, wherein the small molecule is converted via a peptidomimetic chemical approach.

7. A composition for treating or preventing an inflammation comprising the IKK2 inhibitor of claim 1.

8. A method of treating or preventing an inflammation in a patient in need comprising administering to the patient an effective amount of the IKK2 inhibitor of claim 1.

9. The method of claim 8, wherein the IKK2 inhibitor is formulated in a pharmaceutically acceptable carrier or excipients for administration, alone or in combination with one or more agent.

10. A method of treating or preventing an inflammation in a patient in need comprising administering to the patient an effective amount of the composition of claim 7.

11. The method of claim 10, wherein the composition is formulated in a pharmaceutically acceptable carrier or excipients for administration, alone or in combination with one or more agent.

12. A kit comprising
a. at least one IKK2 inhibitor of claim 1; and
b. instructions for treating or preventing inflammation in a patient in need thereof.

13. The kit of claim 12, wherein the IKK2 inhibitor is formulated in a pharmaceutically acceptable carrier or excipients for administration, alone or in combination with one or more agent.

14. A kit comprising
a. at least one composition of claim 7; and
b. instructions for treating or preventing inflammation in a patient in need thereof.

15. The kit of claim 14, wherein the composition is formulated in a pharmaceutically acceptable carrier or excipients for administration, alone or in combination with one or more agent.

16. The composition of claim 7, wherein the IKK2 inhibitor is a peptide NEMO^{ActPep}comprising six amino acids QRRSPP (SEQ ID NO: 1) corresponding to amino acid residues 384-289 of NEMO or a small molecule converted from the peptide NEMO^{ActPep}.

17. The composition of claim 16, wherein the IKK2 inhibitor is formulated in a pharmaceutically acceptable carrier or excipients for administration, alone or in combination with one or more agent.

18. The method of claim 8, wherein the IKK2 inhibitor is a peptide NEMO^{ActPep}comprising six amino acids QRRSPP (SEQ ID NO: 1) corresponding to amino acid residues 384-289 of NEMO or a small molecule converted from the peptide NEMO^{ActPep}.

19. The method of claim 18, wherein the IKK2 inhibitor is formulated in a pharmaceutically acceptable carrier or excipients for administration, alone or in combination with one or more agent.

20. The kit of claim 12, wherein the IKK2 inhibitor is a peptide NEMO^{ActPep} comprising six amino acids QRRSPP (SEQ ID NO: 1) corresponding to amino acid residues 384-289 of NEMO or a small molecule converted from the peptide NEMO^{ActPep}.
